SEQUENCE LISTING (2 sequences)

SEQ ID NO: 1 - protein, 6 aa, Artificial sequence
  Synuclein repetitive imperfect repeat
KTKEGV

SEQ ID NO: 2 - protein, 4 aa, Artificial sequence
  farnesylation sequence
CKAA

(19) United States
(12) Patent Application Publication
(10) Pub. No.: US 2010/0160372 A1
(43) Pub. Date: Jun. 24, 2010

(54) TREATMENT OF PROTEINOPATHIES USING A FARNESYL TRANSFERASE INHIBITOR

(75) Inventors: Peter T. Lansbury, JR., Brookline, MA (US); Craig J. Justman, Cambridge, MA (US); Tom N. Grammatopoulos, Boston, MA (US); Berkley A. Lynch, Cambridge, MA (US); Zhihua Liu, Chevy Chase, MD (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Link Medicine Corporation, Cambridge, MA (US)

(21) Appl. No.: 12/618,265

(22) Filed: Nov. 13, 2009

Related U.S. Application Data
(60) Provisional application No. 61/121,373, filed on Dec. 10, 2008.
(60) Provisional application No. 61/114,219, filed on Nov. 13, 2008.

(51) Int. Cl. A61K 31/4709; A61P 27/02; A61P 29/00; A61P 35/00; A61P 9/00; A61P 25/28; A61P 25/16

(52) U.S. Cl. 514/312

(57) ABSTRACT

Methods and pharmaceutical compositions comprising a low dose of a farnesyl transferase inhibitor useful in the treatment of proteinopathies are provided. These low doses are below the doses used in oncological treatments for which these compounds were initially designed. The treatment includes administering to a subject in need thereof a therapeutically effective amount of a farnesyl transferase inhibitor, wherein the amount is effective to inhibit the farnesylation of a non-Ras FTase substrate involved in the autophagy pathway without substantially affecting the farnesylation of Ras or other oncology related substrates. Treatments in accordance with the present invention may also include an acetylcholinesterase inhibitor, an activator of neurotrophic receptors, an NMDA antagonist, an amyloid deposit inhibitor, an antipsychotic agent, an antidepressant, an anxiolytic, or an antioxidant.

### RELATED APPLICATIONS

This non-provisional patent application claims priority under 35 U.S.C. §119(e) to U.S. Provisional Patent Application Ser. Nos. 61/121,373, filed Dec. 10, 2008, and 61/114,219, filed Nov. 13, 2008, each of which is herein incorporated by reference in its entirety.

### FIELD OF THE INVENTION

The present invention relates to a dosing regimen for using selected farnesyl transferase inhibitors in the treatment of proteinopathies, particularly neurodegenerative diseases including Parkinson's Disease, diffuse Lewy body disease, multiple system atrophy (MSA—the nomenclature initially included three distinct terms: Shy-Drager syndrome, striatonigral degeneration (SD), and olivopontocerebellar atrophy (OPCA)), pantothenate kinase-associated neurodegeneration (e.g., PANK1), cognitive impairment, dementia, amyotrophic lateral sclerosis (ALS), Huntington's Disease (HD), and Alzheimer's Disease (AD) and including other abnormal protein metabolism or accumulation implicated in other pathological disorders such as depression, anxiety, lysosomal storage disease, immune disease, mitochondrial disease, ocular disease, inflammatory disease, cardiovascular disease, or proliferative disease.

### BACKGROUND OF THE INVENTION

A proteinopathy is a disease, disorder, or dysfunction in which abnormal protein metabolism or accumulation has been implicated. Some proteinopathies may include neurodegenerative diseases, cognitive impairment, lysosomal storage diseases, immunologic diseases, mitochondrial diseases, ocular diseases, inflammatory diseases, cardiovascular diseases, and proliferative diseases, etc. Further, included under the umbrella definition of proteinopathies are such specific pathologies as synucleinopathies, tauopathies, amyloidopathies, TDP-43 proteinopathies and others.

Synucleinopathies are a diverse group of neurodegenerative disorders that share a common pathologic lesion containing abnormal aggregates of α-synuclein protein in selectively vulnerable populations of neurons and glia. Certain evidence links the formation of either abnormal filamentous aggregates and/or smaller, soluble pre-filamentous toxic aggregates to the onset and progression of clinical symptoms and the degeneration of affected brain regions in neurodegenerative disorders including Parkinson's disease (PD), diffuse Lewy body disease (DLBD), multiple system atrophy (MSA), and disorders of brain iron concentration including pantothenate kinase-associated neurodegeneration PANK1). The current treatment options for these diseases include symptomatic medications such as carbidopa-levodopa, anticholinergics, and monoamine oxidase inhibitors, with widely variable benefit. Even for the best responders, i.e., patients with idiopathic Parkinson's disease, an initial good response to levodopa is typically overshadowed by drug-induced complications such as motor fluctuations and debilitating dyskinesia, following the first five to seven years of therapy. For the rest of the disorders, the current medications offer marginal symptomatic benefit. Given the severe debilitating nature of these disorders and their prevalence, there is a clear need in the art for novel approaches towards treating and managing synucleinopathies.

Cognitive impairment and dementia are other neurological conditions that are very prevalent and can be debilitating. Cognitive impairment and dementia may be caused by a variety of factors and disease conditions. For example, cognitive impairment or dementia may be caused by atherosclerosis, stroke, cerebrovascular disease, vascular dementia, multi-infarct dementia, Parkinson's disease and Parkinson's disease dementia, Lewy body disease, Pick's disease, Alzheimer's disease, mild cognitive impairment, Huntington's disease, AIDS and AIDS-related dementia, brain neoplasms, brain lesions, epilepsy, multiple sclerosis, Down's syndrome, Rett's syndrome, progressive supranuclear palsy, frontal lobe syndrome, schizophrenia, traumatic brain injury, post coronary artery by-pass graft surgery, cognitive impairment due to electroconvulsive shock therapy, cognitive impairment due to chemotherapy, cognitive impairment due to a history of drug abuse, attention deficit disorder (ADD), attention deficit hyperactivity disorder (ADHD), autism, dyslexia, depression, bipolar disorder, posttraumatic stress disorder, apathy, myasthenia gravis, cognitive impairment during waking hours due to sleep apnea, Tourette's syndrome, autoimmune vasculitis, systemic lupus erythematosus, polymyalgia rheumatica, hepatic conditions, metabolic diseases, Kufs' disease, adrenoleukodystrophy, metachromatic leukodystrophy, storage diseases, infectious vasculitis, syphilis, neurosyphilis, Lyme disease, complications from intracerebral hemorrhage, hypothyroidism, B12 deficiency, folic acid deficiency, niacin deficiency, thiamine deficiency, hydrocephalus, complications post anoxia, prion disease (Creutzfeldt-Jakob disease), Fragile X syndrome, phenylketonuria, malnutrition, and neurofibromatosis, maple syrup urine disease, hypercalcemia, hypothyroidism, and hypoglycemia. Dementia is commonly defined as a progressive decline in cognitive function due to damage or disease in the body beyond what is expected from normal aging. Dementia is described as a loss of mental function, involving problems with memory, reasoning, attention, language, and problem solving. Higher level functions are typically affected first. Dementia interferes with a person's ability to function in normal daily life.

Inclusion body myopathy with early-onset Paget disease and frontotemporal dementia (IBMPFD) is a condition that can affect the muscles, bones, and brain. The first symptom of IBMPFD is often muscle weakness (myopathy), which typically appears in mid-adulthood. Weakness first occurs in muscles of the hips and shoulders, making it difficult to climb stairs and raise the arms above the shoulders. As the disorder progresses, weakness develops in other muscles in the arms and legs. Muscle weakness can also affect respiratory and heart (cardiac) muscles, leading to life-threatening breathing difficulties and heart failure.

Alzheimer's disease (AD) is the leading cause of dementia and cognitive impairment in the elderly and a leading cause of death in developing nations after cardiovascular disease, cancer, and stroke. Up to 70% of cases of dementia are due to Alzheimer's disease, with vascular disease being the second most common cause. The frequency of AD among 60-year-olds is approximately 1%. The incidence of AD doubles approximately every 5 years. Forsyth, Phys. Ther. 78:1325-1331, 1998; Evans et al., JAMA 262:2551-2556, 1989; each of which is incorporated herein by reference. AD afflicts an estimated four million people in the U.S. alone at a cost of $100 billion per year. Schumock, J. Health Syst. Pharm. 55(52):17-21, 1998; Hay & Ernst, Am. J. Public Health 77:1169-1175, 1987; each of which is incorporated herein by reference.

Treatment of cognitive impairment and dementia may be divided into three main areas: pharmacologic interventions targeting the specific underlying pathophysiology; pharmacological agents that ameliorate specific symptoms; and behavioral interventions. The only successful treatments of cognitive impairment in AD to date have been symptomatic treatments such as acetyl cholinesterase inhibitors (e.g., tacrine, donepezil, rivastigmine, and galantamine) and NMDA antagonists (e.g., memantine). There remains a need for other pharmacologic approaches in the treatment of proteinopathies.

### SUMMARY OF THE INVENTION

The present invention stems from recent discoveries in the use of a low dose of a farnesyl transferase inhibitor (FTI) to treat a proteinopathy (e.g., neurodegenerative diseases such as Parkinson's Disease, diffuse Lewy body disease, multiple system atrophy, pantothenate kinase-associated neurodegeneration (e.g., PANK1)) or other neurological condition (e.g., cognitive impairment). One class of proteinopathy diseases is the synucleinopathies, where toxic levels of the protein, alpha-synuclein, accumulates causing a spectrum of diseases and/or disorders. Other diseases where abnormal synuclein metabolism or accumulation has been implicated such as other neurodegenerative diseases such as amyotrophic lateral sclerosis (ALS), Huntington's Disease (HD), and Alzheimer's Disease (AD); cognitive impairment, mitochondrial diseases, ocular diseases, inflammatory diseases, cardiovascular diseases, and proliferative diseases, etc. may also be treated with a low dose of a farnesyl transferase inhibitor based on the present invention. Other proteinopathies, including multiple neurodegenerative diseases with a variety of primary toxic protein pathologies may also be treated as described, as may proteinopathies that lend to diseases of peripheral, non-CNS organs and tissues.

Farnesyl transferase inhibitors of the invention are a compound selected from:

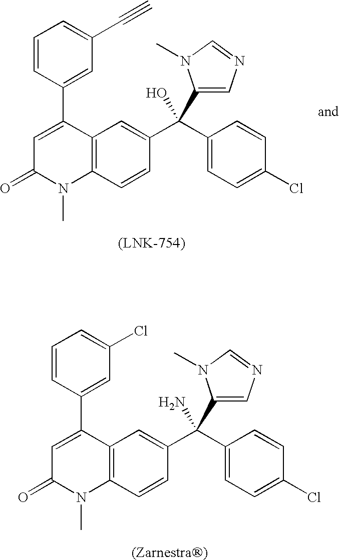

or a salt thereof.

Farnesyl transferase inhibitors were originally developed to inhibit the farnesylation of the Ras protein, which regulates cell proliferation and differentiation and is thus a therapeutic target in treating cancers. In cancer cells, maximal inhibition of the farnesylation of Ras results in cell death. Ras is a member of a broader family of CaaX-CO₂H proteins (where “a” is an amino acid with an aliphatic side chain), all of which are farnesylated at the cysteine residue four amino acid residues from the C-terminus. It has been necessary to use high doses of FTIs to achieve therapeutic efficacy in treating cancers in both animal models and in humans. Such high dose ranges are required to both target the class of CaaX-CO₂H farnesyl transferase substrate proteins like Ras and to achieve a high level of suppression of farnesylation in Ras and related proteins, required for efficacy against cancers. For instance, evidence from animal models shows that Ras farnesylation must be suppressed by at least 50% on average to begin to show toxicity in tumor cells (FIG. 3). Phase I clinical results of both Zarnestra® and LNK-754 indicate that high doses are required to achieve efficacy in treating cancer. Specifically, the recommended Zarnestra® dose for phase II/III testing following a phase I clinical and pharmacological study using continuous dosing was 300 mg twice daily i.e., 600 mg per day (See, Crul, M., et al. Journal of Clinical Oncology, vol. 20, no. 11, 2002, 2726); the recommended phase II dose schedule from another Zarnestra® phase I trial in advanced cancer was 500 mg twice a day i.e., 1000 mg per day (See, Zujewski, J., et al. J. Clin. Oncol. 18:927-941, 2000; and the advised dose from another Zarnestra® phase I trial with patients having advanced leukemia was 600 mg twice a day i.e., 1200 mg per day (See, Ryan, D. P., et al. Proc. Am. Clin. Oncol. 19:185a, 2000). Similarly, a Phase I study of LNK-754 in patients with advanced malignant tumors indicated that a dose of 640 mg twice daily i.e., 1280 mg per day is considered to be slightly less than the dose needed to be clinically effective against ras-expressing tumors (See, Moulder, S. L., et al. Clinical Cancer Research, vol. 10, 2004, 7127-7135).

In addition to the classical farnesyl transferase substrates such as Ras that have the CaaX sequence, there appear to be a class of non-canonical protein substrates that can also be farnesylated by farnesyl transferase (FTase). An example of these proteins is ubiquitin C-terminal esterase L1 (UCH-L1), which has the C-terminal sequence CKAA (where A is alanine). UCH-L1 is a protein expressed in terminally differentiated cells, such as neurons, and which has quite different kinetics of farnesylation than Ras and other CaaX-CO₂H proteins. As a result, it appears that farnesylation of UCH-L I and/or other non-CaaX—CO₂H proteins by FTase can be inhibited by FTIs at much lower concentrations of FTIs than required to inhibit the farnesylation of Ras and related CaaX-CO₂H proteins.

Without wishing to be bound by any particular theory, it is thought that the farnesylation of UCH-L1 and/or other non-CaaX—CO₂H FTase substrates involved in protein clearance pathways are possible targets involved in the treatment of proteinopathies. Therefore, the therapeutically effective amount of an FTI, such as LNK-754 or Zarnestra® or a salt thereof, needed to treat a patient with a proteinopathy would only be the amount needed to inhibit the farnesylation of non-CaaX—CO₂H FTase substrates (e.g., UCH-L1). These doses are much lower than those used to effectively inhibit tumor growth in oncology applications. Having proposed the that the target for the treatment of proteinopathies is possibly UCH-L1 or possibly other non-CaaX—CO₂H FTase substrates, the dosing of LNK-754 or Zarnestra® or a salt thereof, can be tailored to inhibit the farnesylation of non-CaaX-CO₂H proteins without substantially affecting the farnesylation of Ras. In such a way, the side effects associated with the inhibition of the farnesylation of Ras and/or high dose FTI administration may be avoided or at least decreased. Surprisingly, inhibition of the farnesylation of UCH-L1 and other non-CaaX—CO₂H FTase substrates takes place at LNK-754 and Zarnestra® concentrations 5-fold, 10-fold, 50-fold, or even 100-fold lower than those concentrations needed to therapeutically inhibit tumor growth, which is thought to be dependent on the farnesylation of Ras, in the treatment of cancer. Therefore, the inhibition of the farnesylation of UCH-L1 and other non-CaaX—CO₂H FTase substrates may be effected by administering approximately 0.1 mg per day to approximately 150 mg per day, in particular 0.1 mg per day to approximately 50 mg per day, more particularly, approximately 0.5 mg per day to approximately 30 mg per day, more particularly approximately 4 mg per day to approximately 20 mg per day. Since the farnesylation of UCH-L1 and other non-CaaX-CO₂H FTase substrates is inhibited by the FTI, an FTI with the ability to inhibit the farnesylation of a protein (i.e., inhibitors of farnesyl transferase (FTase)) without inhibiting the geranylgeranylation of a protein is particularly useful in the present invention. FTIs with dual activity are associated with greater toxicity as compared to FTase specific inhibitors.

Further, the effect seen by lower concentrations or doses of an FTI may be brought about through a non-farnesylated substrate mechanism. Thus, the effect of the lower concentrations or doses of an FTI may be an interaction of the FTI alone with one or more intracellular protein/s to affect a biochemical/physiological pathway involved in a proteinopathy. Similarly, the effect seen by lower concentrations or doses of an FTI may be brought about through an interaction of the FTI with FTase and with one or more intracellular protein/s to affect a biochemical/physiological pathway involved in a proteinopathy.

It has been discovered that such high doses of FTIs used to treat cancer are not particularly useful in the treatment of other conditions, such as the treatment of proteinopathies. For example, high doses (45 mg/kg) of the FTI, LNK-754, did not significantly lower the number of α-synuclein positive neurons in the brains of treated Masliah D-line transgenic α-synuclein mice (FIG. 2A); however, mice treated with lower doses (0.09 mg/kg to 9 mg/kg) of LNK-754 did show a significant reduction. See FIGS. 2A and 217. Lower doses of LNK-754 (below those doses found to be efficacious in mouse models of cancer) have unexpectedly been found to be useful in the treatment of neurological conditions. The efficacy of FTIs, such as LNK-754 or Zarnestra® or a pharmaceutically acceptable salt thereof, in the treatment of neurological conditions (e.g., Parkinson's disease, Alzheimer's disease) is reduced as the dosing enters that range found to be therapeutically effective in xenograft mouse models of cancer. It is possible that as the FTI begins to significantly inhibit the farnesylation of CaaX—CO₂H proteins at higher doses, it might inhibit pathways that were stimulated by low doses of the FTI. For instance, if inhibition of farnesylation of UCH-L1 stimulates toxic protein clearance by stimulating pathways of protein clearance, such as macroautophagy, inhibition of CaaX-CO₂H protein farnesylation might affect other proteins involved in protein clearance, resulting in an inhibition of protein clearance by high FTI doses.

Further, at lower concentration or doses of an FTI, the interaction of the FTI with other intracellular proteins, with or without FTase involvement, for example acetylation mechanisms of microtubules, may result in a non-farnesylated substrate mechanism of therapeutic treatment of a proteinopathy.

Treatment of α-synuclein transgenic mice with the FTIs, Zamestra® and LNK-754, was found to decrease levels of α-synuclein in the hippocampus, and these mice exhibited fewer α-synuclein inclusions than transgenic animals administered vehicle alone. FIG. 2 shows the efficacy data for LNK-754 in the Masliah D-line transgenic α-synuclein mouse model for synucleinopathies. One trial was performed at the higher doses of 45 mg/kg and 9 mg/kg LNK-754. See FIG. 2A. The higher dose of 45 mg/kg LNK-754 was not found to significantly lower the number of α-synuclein-positive neurons in the brains of treated mice. However, surprisingly the lower dose (9 mg/kg LNK-754) was found to significantly lower the number of α-synuclein-positive neurons in the brains of treated mice. Based on this discovery, a second lower dose trial was performed using doses as low as 0.09 mg/kg and extending to 9 mg/kg. See FIG. 2B. Notably, the doses of LNK-754 used in the second trial were all below the doses found efficacious in mouse models of cancer, but the lowest doses in this trial, 0.9 and 0.09 mg/kg, significantly lowered the number of α-synuclein positive neurons in the transgenic animals.

The invention provides a compound or a pharmaceutically acceptable salt thereof for use in a method of treating a proteinopathic subject, the method comprising administering the compound selected from:

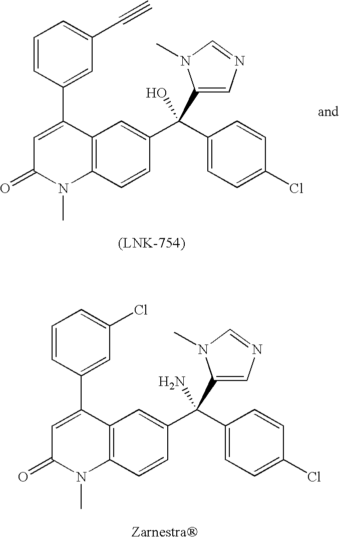

or a pharmaceutically acceptable salt thereof, to the subject in an amount that ranges from approximately 0.1 mg per day to approximately 50 mg per day. In another aspect, the invention provides the use of a compound or a pharmaceutically acceptable salt thereof in the manufacture of a medicament for treating a proteinopathic subject, wherein the medicament comprises a compound or a pharmaceutically acceptable salt thereof selected from LNK-754 and Zarnestra® and the amount of the compound or pharmaceutically acceptable salt thereof administered to the subject ranges from approximately 0.1 mg per day to approximately 50 mg per day. The invention provides a method of treating a proteinopathic subject, wherein the method comprises administering a compound selected from LNK-754 or Zarnestra® or a pharmaceutically acceptable salt thereof, to the subject in an amount that ranges from approximately 0.1 mg per day to approximately 50 mg per day.

The invention provides a compound or pharmaceutically acceptable salt thereof for use in a method of treating a proteinopathic subject, wherein the method comprises administering to the subject an amount of LNK-754 or Zarnestra® or a pharmaceutically acceptable salt thereof, that ranges from approximately 0.5 mg per day to approximately 30 mg per day. The invention provides a method for treating a proteinopathic subject, wherein the amount the compound or a pharmaceutically acceptable salt thereof, ranges from approximately 0.5 mg per day to approximately 30 mg per day.

The invention provides a compound or pharmaceutically acceptable salt thereof for use in a method of treating a proteinopathic subject, wherein the method comprises administering to the subject an amount of LNK-754 or Zarnestra® or a pharmaceutically acceptable salt thereof, that ranges from approximately 4 mg per day to approximately 20 mg per day. The invention provides a method of treating a proteinopathic subject, wherein the amount of the compound or a pharmaceutically acceptable salt thereof, ranges from approximately 4 mg per day to approximately 20 mg per day.

The invention provides a compound or pharmaceutically acceptable salt thereof for use in a method of treating a proteinopathic subject, wherein the method comprises administering to the subject an amount of LNK-754 or Zarnestra® or a pharmaceutically acceptable salt thereof, that is not sufficient to inhibit the farnesylation of Ras in the brain by more than about 50%. The invention provides a method of treating a proteinopathic subject, wherein the amount of the compound or a pharmaceutically acceptable salt thereof, is not sufficient to inhibit the farnesylation of Ras in the brain by more than about 50%.

The invention provides a compound or pharmaceutically acceptable salt thereof for use in a method of treating a proteinopathic subject, wherein the method comprises administering to the subject an amount of LNK-754 or Zarnestra® or a pharmaceutically acceptable salt thereof, that is sufficient to inhibit the farnesylation of UCH-L1. The invention provides a method for treating a proteinopathic subject, wherein the amount of the compound or a pharmaceutically acceptable salt thereof, is sufficient to inhibit the farnesylation of UCH-L1.

The invention provides a compound or pharmaceutically acceptable salt thereof for use in a method of treating a proteinopathic subject, wherein the method comprises administering to the subject the pharmaceutically acceptable D-tartrate salt of LNK-754. The invention provides a method of treating a proteinopathic subject, wherein the method comprises administering to the subject the pharmaceutically acceptable D-tartrate salt of LNK-754.

The invention provides a compound or pharmaceutically acceptable salt thereof for use in a method of treating a proteinopathic subject, wherein the proteinopathic subject is suffering from a neurodegerative disease, a cognitive impairment, a lysosomal storage disease, an ocular disease, an inflammatory disease, a cardiovascular disease, or a proliferative disease. The invention provides a method of treating a proteinopathic subject suffering from neurodegenerative disease. In one aspect, the neurodegenerative disease is selected from Parkinson's disease, diffuse Lewy body disease, multiple system atrophy, pantothenate kinase-associate neurodegeneration, amyotrophic lateral sclerosis, Huntington's disease, and Alzheimer's disease.

The invention provides a compound or a pharmaceutically acceptable salt thereof for use in a method of treating a proteinopathic subject, wherein the method of treating further comprises administering to the subject a compound selected from LNK-754 or Zarnestra® or a pharmaceutically acceptable salt thereof and a therapeutically effective amount of a non-farnesyl transferase inhibitor. The invention provides a method of treating a proteinopathic subject, wherein the method further comprises administering to the subject a compound selected from LNK-754 or Zarnestra® or a pharmaceutically acceptable salt thereof and a therapeutically effective amount of a non-farnesyl transferase inhibitor.

The invention provides the use of a compound or a pharmaceutically acceptable salt thereof in the manufacture of a medicament for treating a proteinopathic subject, wherein the medicament comprises LNK-754 or Zarnestra® or pharmaceutically acceptable salt and a therapeutically effective amount of a non-farnesyl transferase inhibitor.

The invention provides a compound or a pharmaceutically acceptable salt thereof for use in a method of treating a proteinopathic subject, wherein the non-farnesyl transferase inhibitor is selected from the group consisting of dopamine agonists, DOPA decarboxylase inhibitors, dopamine precursors, monoamine oxidase blockers, cathechol O-methyl transferase inhibitors, anticholinergics, acetylcholinesterase inhibitors, activators of neurotrophic receptors, gamma-secretase inhibitors, PDE10 inhibitors, and NMDA antagonists.

The invention provides a compound or a pharmaceutically acceptable salt thereof for use in a method of treating a proteinopathic subject, wherein the subject is a human. The invention provides a method of treating a proteinopathic subject, wherein the subject is human.

The invention provides a pharmaceutical composition for treating a proteinopathic subject, wherein the composition comprises approximately 0.1 mg to approximately 50 mg of a compound selected from LNK-754 or Zarnestra® or a pharmaceutically acceptable salt thereof, and a pharmaceutically acceptable excipient.

The invention provides a pharmaceutical composition, wherein the compositions further comprises approximately 0.5 to approximately 30 mg of LNK-754 or Zarnestra® or a pharmaceutically acceptable salt thereof. The invention provides a pharmaceutical composition, wherein the composition further comprises approximately 4 to approximately 20 mg of LNK-754 or Zarnestra® or a pharmaceutically acceptable salt thereof.

The invention provides a pharmaceutical composition, wherein the composition comprises the pharmaceutically acceptable D-tartrate salt of LNK-754.

The invention provides a pharmaceutical composition for treating a proteinopathic subject, wherein the proteinopathic subject is suffering from a neurodegerative disease, a cognitive impairment, a lysosomal storage disease, an ocular disease, an inflammatory disease, a cardiovascular disease, and a proliferative disease. The invention provides a pharmaceutical composition for treating a proteinopathic subject suffering from a neurodegenerative disease, wherein the neurodegenerative disease is selected from Parkinson's disease, diffuse Lewy body disease, multiple system atrophy, pantothenate kinase-associate neurodegeneration, amyotrophic lateral sclerosis, Huntington's disease, and Alzheimer's disease.

### BRIEF DESCRIPTION OF THE DRAWINGS

FIG. 1 shows the efficacy of LNK-754-TS in a mouse model for cancer. Dosing for 10 days BID in a 3T3H-ras (61 L) xenograft athymic mouse model demonstrates that at least 25 mg per kg of LNK-754-TS per kilogram of body weight are required for suppression of tumor growth in the mouse. From Pfizer Investigational New Drug Application for CP-609,754, Section 8, Pharmacology and Toxicology, dated Nov. 19, 1999. See also Moulder et al., Clinical Cancer Research 10:7127-7135, Nov. 1, 2004.

FIG. 2 shows the efficacy of LNK-754-TS in a mouse model of synucleinopathies (Masliah line-D α-synuclein transgenic mouse). A. Trial of higher doses of LNK-754-TS, 45 mg/kg and 9 mg/kg. Dosing is PO, BID, for 3 months. B. Trial of lower doses of LNK-754-TS. Dosing is PO, BID, for 3 months. LNK-754-TS was found to be efficacious at 9 mg/kg and below. Graphs represent the number of α-synuclein positive cells in the hippocampus of 9 month old α-synuclein transgenic mice. Saline-treated mice feature an age-dependent increase of pathology if compared to baseline mice. All applied dosages of LNK-754-TS led to a significant decrease of the number of α-synuclein IR cells, except for the 9 mg/kg group, in which the significance level was not reached. Data are shown as mean+SEM. # p<0.05 vs. baseline; *P<0.05, **P<0.01 vs. saline.

FIG. 3 provides pharmacokinetic and pharmacodynamic data for continuously infused LNK-754 (CP-609,754) in a 3T3H-ras (61 L) xenograft tumor-bearing athymic mouse (7 day treatment). At continuous serum levels above 100 ng/mL and at least 50% inhibition of Ras farnesylation, significant inhibition of tumor growth was seen. From Pfizer Investigational New Drug Application for CP-609,754, Section 8, Pharmacology and Toxicology, dated Nov. 19, 1999. See also Moulder et al., Clinical Cancer Research 10:7127-7135, Nov. 1, 2004.

FIG. 4 shows relative levels of LC3 mRNA in SH—SY5Y cells on treatment for 72 hours with increasing amounts of LNK-754-TS and with Zarnestra® and Rapamycin.

FIG. 5 demonstrates that LNK-754-TS treatment of SH—SY5Y cells resulted in different dose-response curves for the inhibition of the farnesylation of the Ras versus HDJ2. Samples were derived from the same experiment.

FIG. 6 is a gel that shows the effect of low dose LNK-754-TS treatment on soluble/cytoplasmic Ras level in frontal cortex of alpha-synuclein transgenic mice.

FIG. 7 is a graph that shows the effect of low dose LNK-754-TS treatment on soluble/cytoplasmic Ras level in frontal cortex of alpha-synuclein transgenic mice, and is a quantitation of the data from the gel in FIG. 6.

FIG. 8a is a bar graph that shows that LC3 mRNA is increased by treatment of SH—SY5Y cells with LNK-754-TS (0.01-100 nM), tipifarnib (Zarnestra®; 100 nM), and rapamycin (1 μM) for 72 hr. Data are represented as mean±SEM (n≧5), with statistical significance by ANOVA with Newmans-Kuels post hoc test, annotated as (*) p≦0.05, (**) p≦0.01 and (***) p≦0.001 as compared to control.

FIG. 8b shows punctuate LC3 immunostaining is increased in SH—SY5Y cells treated with LNK-754-TS (100 nM), tipifarnib (Zarnestra®; 100 nM) and rapamycin (1 μM). Cell nuclei are counter stained with DAPI (Scale bar 50 μm).

FIG. 8c is a gel that shows that LC3-II protein level is increased by treatment of SH—SY5Y cells with LNK-754-TS (100 nM) in the presence of Bafilomycin A1 (10 nM). Data are represented as mean+/−SEM with statistical significance by paired student's t-test (n=4, p<0.05).

FIG. 8d is a bar graph that shows mRNA levels of a set of autophagy-related genes that are unaffected by LNK-754-TS (100 nM) and tipifarnib (Zarnestra®; 100 nM), whereas Rapamycin (1 μM) causes upregulation of the autophagy transcript for Atg1, which is downstream of mTOR (which rapamycin acts through). Data are represented as mean±SEM (n≧5), with statistical significance by ANOVA with Newmans-Kuels post hoc test, annotated as (*) p≦0.05, (**) p≦0.01 and (***) p≦0.001 as compared to control.

FIG. 8e is a bar graph that shows p62 mRNA is increased by LNK-754-TS (100 nM) treatment. Data are represented as mean±SEM (n≧5), with statistical significance by ANOVA with Newmans-Kuels post hoc test, annotated as (*) p≦0.05, (**) p≦0.01 and (***) p≦0.001 as compared to control.

FIG. 8f is a gel that shows that Rapamycin (10 nM-10 μM) (but not LNK-754-TS) caused an m-TOR dependent decrease in p70S6K phosphorylation.

FIG. 9a is a pair of graphs that show treatment for three months at two different doses of LNK-754-TS (0.9 mg/kg (n=8) and 0.09 mg/kg (n=9), twice every 24 hr) halts deposition in both cortex and hippocampus.

FIG. 9b is a graph that shows treatment of transgenic α-synuclein overexpressing mice for three months with LNK-754-TS (2 mg/kg (n=9) once every 72 hr). In this experiment, the mice have high baseline (before beginning treatment) levels of cortical α-synuclein accumulation and do not progress during the course of treatment (baseline vs. vehicle). However, treatment with LNK-754-TS, significantly reduces α-synuclein immunoreactivity below baseline and vehicle treated controls.

FIG. 9c is a series of images that show representative hippocampal slices (reduction of immunoreactivity is ca. 50%) from a three-month dosing trial demonstrating a clear reduction of α-synuclein (green) in cell bodies and in the neuropil, and lack of effect on neuronal architecture (red=NeuN). Data are represented as mean±SEM and statistical significance by ANOVA with Newman-Kuels post hoc test is annotated as (*) p≦0.05, and (***) p≦0.001 as compared to vehicle group.

FIG. 10a is a graph that shows Tau immunoreactivity, as measured by immunostaining with two different antibodies (phosphorylated-Tau with the antibody AT180 and total-Tau with the antibody HT7), increased in transgenic mouse brain over three months (baseline vs. vehicle-treated). Three month treatment of LNK-754-TS (0.09 mg/kg (n=6), once every 24 hours) significantly reduced P-Tau (AT180) immunoreactivity but did not change total Tau (HT7) levels.

FIG. 10b is a series of two graphs that show LNK-754-TS treatment (0.09 mg/kg (n=6), once every 24 hr) significantly increased struggling and decreased floating to levels equivalent to that seen in non-transgenic mice. Data are represented as mean±SEM with statistical significance by ANOVA repeated measure with either Newman-Kuels (for a) or Dunnett post hoc test, annotated as (*) p≦0.05, (**) p≦0.01 and (***) p≦0.001 as compared to vehicle group.

FIG. 11a is a graph that shows LNK-754-TS treatment (0.9 mg/kg (n=5), once every 24 hours) in an APP/PS1 transgenic mouse model of alzheimer's disease (having elevated levels of brain A-beta 1-42) caused a significant cognitive improvement after two months of dosing when compared to vehicle group.

FIG. 11b is a series of two bar graphs that show LNK-754-TS treatment (0.9 mg/kg (n=5), once every 24 hr) in the same APP/PS1 experiment as FIG. 11a showed a significant decrease in the number of Aβ plaques (grey bars) in the area of the subiculum when compared to vehicle. Data are represented as Mean±SEM with student T test statistical significance p≦0.05, annotated as (8).

FIG. 11c is a graph that shows in a second study, but in the same APP/PS1 transgenic mice, there is cognitive improvement after 12 days of dosing with LNK-754-TS (0.9 mg/kg (n≧20), once every 24 hours) when compared to vehicle group. Nontransgenic animals were also tested (black circles). Data are represented as mean±SEM and statistical significance by ANOVA repeated measure with Dunnett post hoc test is annotated as (*) p≦0.05, (**) p≦0.01 and (***) p≦0.001 as compared to vehicle group.

FIG. 12 is a graph that shows the pharmacokinetic profile of LNK-754-TS in WT mice in plasma and brain after a single dose of either 9 mg/kg or 0.9 mg/kg

FIG. 13 is a graph that shows the pharmacokinetic profile of Zarnestra® in C57BL/6 mice when administered at 5 mg/kg, 20% beta-cyclodextrin, p.o., single dose. LLOQ: brain 4 ng/g; plasma 50 ng/ml.

FIG. 14 is a graph that shows the inhibition of FTase within human peripheral blood mononuclear cells at Cₘₐₓ (2 hours after a single oral administration of LNK-754-TS at various doses).

### DEFINITIONS

As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “farnesyl transferase inhibitor” generally refers to any compound that inhibits the farnesylation of a protein known to be farnesylated in viva. In particular, a farnesyl transferase inhibitor specifically inhibits a farnesyl transferase (FTase). The farnesyl transferase inhibitor preferably does not substantially inhibit geranylgeranyl transferase (GGTase). In certain embodiments, the farnesyl transferase inhibitor inhibits the farnesylation of UCH-L1. In certain embodiments the farnesyl transferase inhibitor activates autophagy or stimulates protein clearance. In certain embodiments, the farnesyl transferase inhibitor inhibits the farnesylation of a protein with a non-CaaX C-terminal farnesylation sequence. In certain embodiments, the farnesyl transferase inhibitor inhibits the farnesylation of a protein with the C-terminal farnesylation sequence —CKAA-CO₂H. In certain embodiments, the dose of the farnyesyl transferase inhibitor can be titrated to inhibit the farnesylation of proteins with non-CaaX farnesylation sequences without inhibiting the farnesylation of Ras or other proteins with the farnesylation sequence —CaaX—CO₂H. In certain embodiments, the dose of the farnesyl transferase inhibitor can be titrated to inhibit the farnesylation of UCH-L1 or other proteins with the farnesylation sequence CKAA-CO₂H without inhibiting the farnesylation of Ras or other proteins with the farnesylation sequence —CaaX—CO₂H. In certain embodiments, the farnesyl transferase inhibitor affects protein aggregation via a non-farnesylated substrate mechanism. The FTI may be involved with interacting with additional intracellular proteins, with or without FTase, to affect biochemical or physiological pathways involved in autophagy or protein clearance.

As used herein, the term “LNK-754” refers to a compound having the structure:

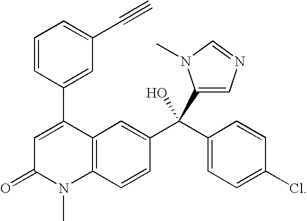

Synonyms include CP 609754, OSI 754, and '754. Alternative chemical names include: (R)-6-[(4-chlorophenyl)-hydroxyl-(1-methyl-1-H-imidazol-5-yl)-methyl]-4-(3-ethynylphenyl)-1-methyl-2-(1H)-quinolinone and (R)-6-[(4-chlorophenyl)-hydroxyl-(3-methyl-3-H-imidazol-4-yl)-methyl]-4-(3-ethynylphenyl)-1-methyl-2-(1H)-quinolinone.

As used herein, the term “LNK-754-TS” means the D-tartrate salt of LNK-754. Alternative chemical names for LNK-754-TS include: (R)-6-[(4-chlorophenyl)-hydroxyl-(1-methyl-1-H-imidazol-5-yl)-methyl]-4-(3-ethynylphenyl)-1-methyl-2-(1H)-quinolinone (2S, 3 S)-dihydroxybutanedioate and (R)-6-[(4-chlorophenyl)-hydroxyl-(3-methyl-3-H-imidazol-4-yl)-methyl]-4-(3-ethynylphenyl)-1-methyl-2-(1H)-quinolinone (2S,3S)-dihydroxybutanedioate.

As used herein, the term “Zarnestra®” refers to a compound having the structure:

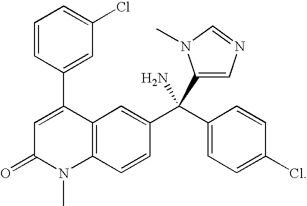

Synonyms include R115777, tipifarnib, and (R)-6-(Amino(4-chlorophenyl)(1-methyl-1H-imidazol-5-yl)methyl)-4-(3-chlorophenyl)-1-methyl-2(1H)-quinolinone.

As used herein, the term “in vitro” refers to events that occur in an artificial environment, e.g., in a test tube or reaction vessel, in cell culture, etc., rather than within an organism (e.g., animal, plant, and/or microbe).

As used herein, the term “in vivo” refers to events that occur within an organism (e.g., animal, plant, and/or microbe).

As used herein, the term “patient” or “subject” refers to any organism to which a composition of this invention may be administered. Typical subjects include animals (e.g., mammals such as mice, rats, rabbits, non-human primates, and humans; insects; worms; etc.).

In one embodiment, the subject is human. In some embodiments, a subject may be suffering from a disease, disorder, and/or condition. In some embodiments, a subject may be susceptible to a disease, disorder and/or condition.

As used herein, the term “proteinopathic subject” refers to a subject that is diagnosed with or affected by, or at risk of developing a proteinopathy (e.g., predisposed, for example genetically predisposed, to developing a proteinopathy) including any disorder characterized by abnormal protein metabolism or accumulation. The term “subject with a proteinopathy” refers to a subject that is diagnosed with or affected by a proteinopathy, including any disorder characterized by abnormal protein metabolism or accumulation. The term “subject at risk of developing a proteinopathy” refers to a person that is predisposed, for example genetically predisposed, to developing a proteinopathy) and/or any disorder characterized by abnormal protein metabolism or accumulation. Proteinopathy includes neurodegenerative diseases, cognitive impairment, lysosomal storage diseases, immunologic diseases, mitochondrial diseases, ocular diseases, and some proliferative diseases. Proteinopathic subjects can be readily identified by persons of ordinary skill in the art by symptomatic diagnosis and neurologic examination and/or in some instances in conjunction with genetic screening, brain scans, SPEC, PET imaging, etc.

In the methods of the invention, the term “proteinopathy” includes neurodegenerative diseases including Parkinson's Disease, diffuse Lewy body disease, multiple system atrophy (MSA—the nomenclature initially included three distinct terms: Shy-Drager syndrome, striatonigral degeneration (SD), and olivopontocerebellar atrophy (OPCA)), pantothenate kinase-associated neurodegeneration (e.g., PANK1), cognitive impairment, dementia, amyotrophic lateral sclerosis (ALS), Huntington's Disease (HD), and Alzheimer's Disease (AD) and includes other abnormal protein metabolism or accumulation implicated in other pathological disorders such as depression, anxiety, lysosomal storage disease, immune disease, mitochondrial disease, ocular disease, inflammatory disease, cardiovascular disease, or proliferative disease.

As used herein, the term “synucleinopathic subject” refers to a subject that is diagnosed with or affected by a synucleinopathy (e.g., predisposed, for example genetically predisposed, to developing a synucleinopathy) and/or any neurodegenerative disorder characterized by pathological synuclein aggregations. Several neurodegenerative disorders including Parkinson's disease, diffuse Lewy body disease (DLBD), multiple system atrophy (MSA), and disorders of brain iron concentration including pantothenate kinase-associated neurodegeneration (e.g., PANK1) are collectively grouped as synucleinopathies. These subjects can be readily identified by persons of ordinary skill in the art by symptomatic diagnosis and neurologic examination and/or in some instances in conjunction with genetic screening, brain scans, SPEC, PET imaging, etc.

The term “subject with a synucleinopathy” refers to a subject that is diagnosed with or affected by a synucleinopathy disorder. The term “subject at risk of developing a synucleinopathy” refers to a person that is predisposed, for example genetically predisposed, to developing a synucleinopathy. Synucleinopathic subjects can be readily identified by persons of ordinary skill in the art by symptomatic diagnosis and neurologic examination and/or in some instances in conjunction with genetic screening, brain scans, SPEC, PET imaging, etc.

In methods of the invention, the term “synucleinopathy” refers to neurological disorders that are characterized by a pathological accumulation of α-synuclein. This group of disorders includes, but is not necessarily limited to, Parkinson's disease, diffuse Lewy body disease (DLBD), multiple system atrophy (MSA), and disorders of brain iron concentration including pantothenate kinase-associated neurodegeneration (e.g., PANK1).

As used herein, the term “protein” refers to a polypeptide (i.e., a string of at least two amino acids linked to one another by peptide bonds). Proteins may include covalently-linked moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence) or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, farnesylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

In general, a “small molecule” is understood in the art to be an organic molecule that is less than about 2000 g/mol in size. In some embodiments, the small molecule is less than about 1500 g/mol or less than about 1000 g/mol. In some embodiments, the small molecule is less than about 800 g/mol or less than about 500 g/mol. In some embodiments, small molecules are non-polymeric and/or non-oligomeric. In some embodiments, small molecules are not proteins, peptides, or amino acids. In some embodiments, small molecules are not nucleic acids or nucleotides. In some embodiments, small molecules are not saccharides or polysaccharides.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

An individual who is “suffering from” a disease, disorder, and/or condition has been diagnosed with and/or displays one or more symptoms of a disease, disorder, and/or condition.

An individual who is “susceptible to” a disease, disorder, and/or condition has not been diagnosed with a disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition (e.g., a proteinopathy).

As used herein, the term “therapeutically effective amount” means an amount of an FTI such as LNK-754 or Zarnestra® or salt thereof, or composition comprising an FTI, that inhibits the farnesylation of UCH-L1 or other farnesylated target without inhibiting the farnesylation of Ras to the extent needed in oncological applications. In certain embodiments, LNK-754 or Zarnestra® or salt thereof inhibits the farnesylation of UCH-L1 by more than about 70%, 80%, 90%, 95%, 97%, 98%, 99%, or 99.9%. In certain embodiments, the therapeutically effective amount of the FTI does not inhibit the farnesylation of Ras by more than 10%, 20%, 30%, 40%, 50%, or 60%. In certain embodiments, the therapeutically effective amount of the FTI does not inhibit the farnesylation of a protein with a farnesylation sequence of —CaaX—CO₂H, wherein C is cysteine, a is an aliphatic amino acid residue, and X is serine, methionine, glutamine, alanine, or threonine, by more than 10%, 20%, 30%, 40%, 50%, or 60%. In certain embodiments, the therapeutically effective amount of LNK-754 or Zamestra® or salt thereof, treating neurological diseases is below therapeutically effective oncological doses of the FTI. In some embodiments, a therapeutically effective amount of a substance is an amount that is sufficient, when administered to a subject suffering from or susceptible to a proteinopathy to treat, diagnose, prevent, and/or delay the onset of the proteinopathy. As will be appreciated by those of ordinary skill in this art, the effective amount of the FTI may vary depending on such factors as the desired biological endpoint, the FTI to be delivered, the disease or condition being treated, the subject be treated, etc.

A therapeutically effective amount of an FTI for treating cancer or for use in ontological applications is that amount of the FTI required to inhibit the farnesylation of Ras to an extent necessary to result in a cytotoxic effect in cancer cells. In certain embodiments, it is the equivalent dose in humans to those observed to be effective in animal models of cancer. In certain embodiments, the therapeutically effective amount of the FTI for use in treating cancer results in at least 50% inhibition of Ras farnesylation.

As used herein, the term “treat,” “treatment,” or “treating” refers to any method used to partially or completely alleviate, ameliorate, relieve, inhibit, reduce severity of, and/or reduce incidence of one or more symptoms or features of a disease, disorder, and/or condition. In some embodiments, treatment may be administered to a subject who exhibits only early signs of the disease, disorder, and/or condition for the purpose of decreasing the risk of developing pathology associated with the disease, disorder, and/or condition.

As used herein, the term “prevent,” “prevention,” or “preventing” refers to any method to partially or completely prevent or delay the onset of one or more symptoms or features of a disease, disorder, and/or condition. Prevention may be administered to a subject who does not exhibit signs of a disease, disorder, and/or condition.

The term stereochemically isomeric forms of compounds, as used herein, include all possible compounds made up of the same atoms bonded by the same sequence of bonds but having different three-dimensional structures which are not interchangeable, which the compounds may possess. Unless otherwise mentioned or indicated, the chemical designation of a compound encompasses the mixture of all possible stereochemically isomeric forms that the compound can take. The mixture can contain all diastereomers and/or enantiomers of the basic molecular structure of the compound. All stereochemically isomeric forms of the compounds either in pure form or in admixture with each other are intended to be embraced within the scope of the present invention.

Some of the compounds may also exist in their tautomeric forms. Such forms although not explicitly indicated in the above formula are intended to be included within the scope of the present invention.

Various forms of “prodrugs” are known in the art. For examples of such prodrug derivatives, see:

Design of Prodrugs, edited by H. Bundgaard, (Elsevier, 1985) and Methods in Enzymology, 42:309-396, edited by K. Widder, et al. (Academic Press, 1985);

A Textbook of Drug Design and Development, edited by Krogsgaard-Larsen;

Bundgaard, Chapter 5 “Design and Application of Prodrugs”, by H. Bundgaard, p. 113-191 (1991);

H. Bundgaard, Advanced Drug Delivery Reviews, 8:1-38 (1992);

H. Bundgaard, et al., Journal of Pharmaceutical Sciences, 77:285 (1988); and

N. Kakeya, et al., Chem. Pharm. Bull., 32:692 (1984).

The methods and structures described herein relating to compounds and compositions of the invention also apply to the pharmaceutically acceptable acid or base addition salts and all stereoisomeric forms of these compounds and compositions.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, the present invention relates to a compound represented by any of the structures outlined herein, wherein the compound is a single stereoisomer.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as anti-proteinopathy farnesyl transferase inhibitor compounds), wherein one or more simple variations of substituents are made which do not adversely affect the efficacy of the compound. The compounds of the present invention may be prepared by the methods illustrated in the reaction schemes described herein, or by modifications thereof, using readily available starting materials, reagents and conventional synthesis procedures. In these reactions, it is also possible to make use of variants, which are in themselves known, but are not mentioned here. The present invention includes a method of synthesizing LNK-754 or a pharmaceutically acceptable salt thereof e.g., the D-tartrate salt.

For purposes of this invention, the chemical elements are identified in accordance with the Periodic Table of the Elements, CAS version, Handbook of Chemistry and Physics, 67th Ed., 1986-87, inside cover.

In another aspect, the present invention provides pharmaceutical compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As set out herein, certain embodiments of the present compounds may contain a basic functional group, such as amino or alkylamino, and are, thus, capable of forming pharmaceutically acceptable salts with pharmaceutically acceptable acids. The term “pharmaceutically acceptable salts” in this respect refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. See, for example, Berge et al. (1977) “Pharmaceutical Salts”, J. Pharm. Sci. 66:1-19; incorporated herein by reference.

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically-acceptable organic primary, secondary or tertiary amine. Appropriate base salt forms include, for example, the ammonium salts, the alkali and earth alkaline metal salts, e.g. the lithium, sodium, potassium, magnesium, calcium salts and the like, salts with organic bases, e.g. the benzathine, N-methyl-D-glutamine, hydrabamine salts, and salts with amino acids such as, for example, arginine, lysine and the like. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. See, for example, Berge et al., supra. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The terms acid or base addition salt also comprise the hydrates and the solvent addition forms which the compounds are able to form. Examples of such forms are e.g. hydrates, alcoholates and the like.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

The phrases “systemic administration,” “administered systemically,” “peripheral administration,” and “administered peripherally” as used herein mean the administration of a compound, drug or other material other than directly into the central nervous system, such that it enters the patient's system and, thus, is subject to metabolism and other like processes, for example, subcutaneous administration.

As used herein, the term “subject with cognitive impairment” refers to a subject that is diagnosed with, affected by, or at risk of developing cognitive impairment. The cognitive impairment may stem from any etiology. Exemplary causes of cognitive impairment include neurodegenerative diseases, neurological diseases, psychiatric disorders, genetic diseases, infectious diseases, metabolic diseases, cardiovascular diseases, vascular diseases, aging, trauma, malnutrition, childhood diseases, chemotherapy, autoimmune diseases, and inflammatory diseases. Particular disease that are associated with cognitive impairment include, but are not limited to, atherosclerosis, stroke, cerebrovascular disease, vascular dementia, multi-infarct dementia, Parkinson's disease and Parkinson's disease dementia, Lewy body disease, Pick's disease, Alzheimer's disease, mild cognitive impairment, Huntington's disease, AIDS and AIDS-related dementia, brain neoplasms, brain lesions, epilepsy, multiple sclerosis, Down's syndrome, Rett's syndrome, progressive supranuclear palsy, frontal lobe syndrome, schizophrenia, traumatic brain injury, post coronary artery by-pass graft surgery, cognitive impairment due to electroconvulsive shock therapy, cognitive impairment due to chemotherapy, cognitive impairment due to a history of drug abuse, attention deficit disorder (ADD), attention deficit hyperactivity disorder (ADHD), autism, dyslexia, depression, bipolar disorder, post-traumatic stress disorder, apathy, myasthenia gravis, cognitive impairment during waking hours due to sleep apnea, Tourette's syndrome, autoimmune vasculitis, systemic lupus erythematosus, polymyalgia rheumatica, hepatic conditions, metabolic diseases, Kufs' disease, adrenoleukodystrophy, metachromatic leukodystrophy, storage diseases, infectious vasculitis, syphillis, neurosyphillis, Lyme disease, complications from intracerebral hemorrhage, hypothyroidism, B12 deficiency, folic acid deficiency, niacin deficiency, thiamine deficiency, hydrocephalus, complications post anoxia, prion disease (Creutzfeldt-Jakob disease), Fragile X syndrome, phenylketonuria, malnutrition, neurofibromatosis, maple syrup urine disease, hypercalcemia, hypothyroidism, hypercalcemia, and hypoglycemia. The degree of cognitive impairment may be assessed by a health care professional. A variety of standardized tests are available for assessing cognition, including, but not limited to, the Mini-Mental Status Examination, the Dementia Symptom Assessment Scale, and the ADAS. Such tests typically provide a measurable score of cognitive impairment.

As used herein, the term “subject with depression” refers to a subject that is diagnosed with, affected by, or at risk of developing depression. Based on the treatment of a transgenic mouse overexpressing Tau with a farnesyl transferase inhibitor, reduced Tau transgene-induced depression was seen in the treated mice indicated by an increase in struggling and decreased floating in the forced swim test as compared to control animals. In addition, FTI-treated mice overexpressing TAU displayed behavior similar to non-transgenic animals. The treated mice also showed reduced phosphorylated TAU in the amygdala.

As used herein, the term “subject with anxiety” refers to a subject that is diagnosed with, affected by, or at risk of developing anxiety. The anxiety may stem from a variety of causes. Based on mouse studies, farnesyl transferase inhibitors may be used as anxiolytics.

### DETAILED DESCRIPTION OF CERTAIN EMBODIMENTS OF THE INVENTION

The present invention provides methods of treatment and pharmaceutical compositions for treating a subject with a proteinopathy using a farnesyl transferase inhibitor at a low dose that does not inhibit the farnesylation of Ras at levels necessary for treating cancer and/or is below doses in humans and other mammals equivalent to the therapeutically effective doses in xenograft mouse models of cancer. Such a low dose of the farnesyl transferase inhibitor reduces the side effects and toxicity associated with inhibiting the farnesylation of Ras and possibly related farnesylated targets. In certain embodiments, the dose of the farnesyl transferase inhibitor selectively inhibits the farnesylation of UCH-L1 to effectively treat a neurological disease without substantially affecting the farnesylation of Ras. It has been found that high doses of FTIs intended to be useful in the treatment of cancer are not efficacious in the treatment of proteinopathies. In contrast, doses below those useful in the treatment of cancer have been found to be efficacious in proteinopathic applications. The effect seen by lower concentrations or doses of an FTI may be brought about through a mechanism not involving inhibition of protein farnesylation. For example, an FTI alone, or an FTI/FTase/farnesyl pyrophosphate or FTI/FTase complex, may interact with one or more intracellular protein/s, including microtubules and HDAC, to affect a biochemical/physiological pathway involved in a proteinopathy. In certain embodiments, the invention provides methods for treating a subject with a proteinopathy. In certain embodiments, the invention provides methods for treating a subject with a prototypic synucleinopathy, such as Parkinson's disease (PD), diffuse Lewy body disease (DLBD), multiple system atrophy (MSA), and pantothenate kinase-associated neurodegeneration (DANK). In other embodiments, the invention provides methods for treating a subject with a neurodegenerative disease, such as amyotrophic lateral sclerosis (ALS), Huntington's disease (HD), or Alzheimer's disease (AD), or other neurological conditions, such as cognitive impairment, depression, or anxiety. Typically, the neurological condition being treated with an FTI is associated with protein aggregation and/or protein accumulation in the cell that leads to toxicity.

Without wishing to be bound by any particular theory or mechanism of action, methods of the invention are useful in inducing protein clearance (e.g., accelerating the clearance and/or degradation of α-synuclein, phospho-Tau, Tau, or intracellular A-beta, the accumulation of which are pathogenic in various neurological conditions). In certain embodiments, the methods of the invention induce autophagy. In certain embodiments, the methods of the invention induce autophagy in neuronal cells. In certain embodiments, the treatment method inhibits the accumulation of α-synuclein or other toxic proteins as a result of stimulating degradation. In other embodiments, the treatment method prevents the aggregation of α-synuclein or other toxic proteins as a result of stimulating degradation. In still other embodiments, the treatment method decreases levels of both soluble and insoluble α-synuclein or other toxic proteins. The invention provides methods for treating a subject with a proteinopathy disease associated with toxic protein accumulation, including the step of administering to the subject an amount of a farnesyl transferase inhibitor e.g., LNK-754 or Zarnestra®, or a composition thereof, effective to inhibit the farnesylation of UCH-L1 or other protein associated with protein clearance pathways without substantially inhibiting the farnesylation of Ras and/or related proteins. In certain embodiments, the amount of the farnesyl transferase inhibitor administered is effective to inhibit the farnesylation of a protein with a farnesylation sequence that does not belong to the CaaX-CO₂H family, such as CKAA-CO₂H, without substantially inhibiting the farnesylation of a protein with a farnesylation sequence of CaaX-CO₂H; wherein C is cysteine, K is lysine, A is alanine, a is an aliphatic amino acid, and X is independently serine, methionine, glutamine, alanine, or threonine. In certain embodiments, rather than determining the farnesylation state of UCH-L1 or other non-CaaX—CO₂H FTase substrates directly, a surrogate marker such as HDJ2 is used in human clinical or animal studies. Optionally, the farnesylation of Ras is determined. In certain embodiments, the subject being treated using the inventive method is a mammal. In certain embodiments, the subject is a human. The human may be male or female, and the human may be at any stage of development. Pharmaceutical compositions comprising LNK-754 or Zarnestra® or salt thereof, for use in accordance with the present invention are also provided.

In one aspect, the invention provides a method of treating a proteinopathy in a subject suffering therefrom, the method comprising administering to a subject an FTI at a low dose that does not substantially affect the farnesylation of Ras and/or is below efficacious doses in a xenograft mouse model of cancer. The proteinopathy may be due to any of a variety of etiologies.

### Farnesyl Transferase Inhibitor

A farnesyl transferase inhibitor specifically inhibits farnesyl transferase (FTase), thereby leading to the inhibition of the farnesylation of one, several or many target protein/s (e.g., Ras, UCH-L1, HDJ2). In certain embodiments, the farnesyl transferase inhibitor used at certain doses inhibits the farnesylation of UCH-L1. In certain embodiments, the farnesyl transferase inhibitor used at certain doses inhibits the farnesylation of a non-CaaX—CO₂H FTase substrate. In certain embodiments, the farnesyl transferase inhibitor used at certain doses inhibits the farnesylation of HDJ2. In certain embodiments, the farnesyl transferase inhibitor may have been developed to inhibit the farnesylation of Ras protein. In certain embodiments, the farnesyl transferase inhibitor does not substantially affect the geranylgeranylation of proteins. For examples, LNK-754 and Zarnestra® have been found to be selective FTase inhibitors, with little to no GGTase inhibitory activity. Greater toxicity has been seen with FTIs that have the dual inhibitory activity (i.e., inhibiting both FTase and GGTase). In general, FTase specific inhibitors are preferred in order to minimize toxicity and other undesired side effects. In certain embodiments, the farnesyl transferase inhibitor, alone or associated with FTase, interacts with one, several or many intracellular proteins that are involved with autophagy or protein clearance pathways.

FTIs inhibit the farnesylation of a target peptide or protein by a farnesyl transferase. The inhibitory activity may be determined by in vivo and/or in vitro assays. The assay may be based on the farnesylation of a particular target protein or peptide (e.g., Ras, HDJ2, UCH-L1, etc.). In certain embodiments, the IC₅₀ as measured in an in vitro assay using a farnesyl transferase (FTase) is less than about 100 nM. In certain embodiments, the IC₅₀ is less than about 50 nM. In certain embodiments, the IC₅₀ is less than about 10 nM. In certain embodiments, the IC₅₀ is less than about 5 nM. In certain embodiments, the IC₅₀ is less than about 1 nM. The farnesyl transferase used in the assay may be a recombinant FTase, purified FTase, partially purified FTase, crude FTase, or FTase activity in cells or tissues.

The farnesyltransferase inhibitors of the invention include the compound:

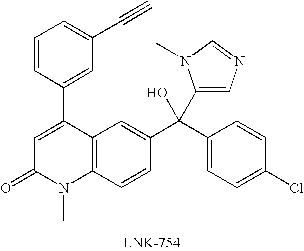

or a pharmaceutically acceptable derivative, pro-drug, analog, stereoisomer, isomer, hydrate, solvate, polymorph, co-crystal, or salt thereof, at a therapeutically effective dose and frequency. In certain embodiments, the tartrate salt of the compound is administered. In certain embodiments, the D-tartrate salt of the compound is administered.

The farnesyltransferase inhibitors of the invention include the compound:

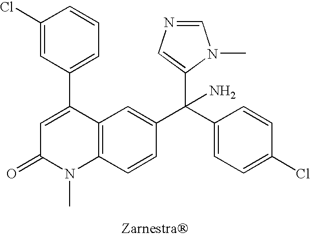

or a pharmaceutically acceptable derivative, pro-drug, analog, stereoisomer, isomer, hydrate, solvate, polymorph, co-crystal, or salt thereof, at a therapeutically effective dose and frequency.

### Uses of FTIs in the Treatment of Proteinopathies and Other Neurological Conditions

As used herein, the term “proteinopathy” refers to diseases, disorders, and/or conditions associated with the pathogenic accumulation and/or aggregation of one or more types of proteins (for example, but not limited to e.g., α-synuclein, amyloid beta proteins, and/or tau proteins). In some embodiments, a proteinopathy may involve pathological alterations in one or more of protein folding, degradation (e.g., autophagy), transportation, etc. Autophagy may include microautophagy, macroautophagy, chaperone-mediated autophagy, mitophagy, pexophagy. Some proteinopathies may include neurodegenerative diseases, some may include cognitive impairment, some may include lysosomal storage diseases, some may include immunologic diseases, some may include mitochondrial diseases, some may include ocular diseases, some may include inflammatory diseases, some may include cardiovascular diseases, and some may include proliferative diseases, etc. Included under the umbrella definition of proteinopathies are such specific pathologies as synucleinopathies, tauopathies, amyloidopathies, TDP-43 proteinopathies and others. Exemplary proteins involved in proteinopathies include: α-synuclein in the case of PD, Lewy body disease, and other synucleinopathies; Tau and Aβ in the case of AD and certain other neurodegenerative diseases; SOD1 and TDP-43 in the case of ALS; huntingtin in the case of Huntington's disease, rhodopsin in the case of retinitis pigmentosa, and a number of proteins in the case of the diseases collectively known as lysosomal storage disease. Indeed, in lysosomal storage diseases, there is often an accumulation of certain lipids eg glucosylceramide or cholesterol, or of certain proteins (e.g., subunit c of ATP synthase), or of certain damaged organelles or organelle fragments e.g., fragmented mitochondria.

### Synucleinopathy

The present invention provides methods related to synucleinopathies. Synucleinopathies are a diverse set of disorders that share a common association with lesions containing abnormal aggregates of α-synuclein protein. Typically such lesions are found in selectively vulnerable populations of neurons and glia. Certain evidence links the formation of either abnormal filamentous aggregates and/or smaller, soluble pre-filamentous toxic aggregates to the onset and progression of clinical symptoms and the degeneration of affected brain regions in neurodegenerative disorders including Parkinson's disease (PD), diffuse Lewy body disease (DLBD), multiple system atrophy (MSA—the nomenclature initially included three distinct terms: Shy-Drager syndrome, striatonigral degeneration (SD), and olivopontocerebellar atrophy (OPCA)), and disorders of brain iron concentration including pantothenate kinase-associated neurodegeneration (e.g., PANK1).

Synucleins are small proteins (123 to 143 amino acids) characterized by repetitive imperfect repeats KTKEGV (SEQ ID NO: 1) distributed throughout most of the amino terminal half of the polypeptide in the acidic carboxy-terminal region. There are three human synuclein proteins termed α, β, and γ, and they are encoded by separate genes mapped to chromosomes 4221.3-q22, 5q23, and 10q23.2-q23.3, respectively. The most recently cloned synuclein protein synoretin has a close homology to γ-synuclein and is predominantly expressed within the retina. α-synuclein, also referred to as non-amyloid component of senile plaques precursor protein (NACP), SYN 1 or synelfin, is a heat-stable, “natively unfolded” protein of poorly defined function. It is predominantly expressed in the central nervous system (CNS) neurons where it is localized to presynaptic terminals. Electron microscopy studies have localized α-synuclein in close proximity to synaptic vesicles at axonal termini, suggesting a role for α-synuclein in neurotransmission or synaptic organization, and biochemical analysis has revealed that a small fraction of α-synuclein may be associated with vesicular membranes but most α-synuclein is cytosolic.

Genetic and histopathological evidence supports the idea that α-synuclein is the major component of several proteinaceous inclusions characteristic of specific neurodegenerative diseases. Pathological synuclein aggregations are restricted to the α-synuclein isoforms, as β and γ synucleins have not been detected in these inclusions. The presence of α-synuclein positive aggregates is disease specific. Lewy bodies, neuronal fibrous cytoplasmic inclusions that are histopathological hallmarks of Parkinson's disease (PD) and diffuse Lewy body disease (DLBD) are strongly labeled with antibodies to α-synuclein. Dystrophic ubiquitin-positive neurites associated with PD pathology, termed Lewy neurites (LN) and CA2/CA3 ubiquitin neurites are also α-synuclein positive. Furthermore, pale bodies, putative precursors of LBs, thread-like structures in the perikarya of slightly swollen neurons and glial silver positive inclusions in the midbrains of patients with LB diseases are also immunoreactive for α-synuclein. α-synuclein is likely the major component of glial cell inclusions (GCIs) and neuronal cytoplasmic inclusions in MSA and brain iron accumulation type 1 (PANK1). α-synuclein immunoreactivity is present in some dystrophic neurites in senile plaques in Alzheimer's Disease (AD) and in the cord and cortex in amyotrophic lateral sclerosis (ALS). α-synuclein immunoreactivity is prominent in transgenic and toxin-induced mouse models of PD, AD, ALS, and HD.

Further evidence supports the notion that α-synuclein is the actual building block of the fibrillary components of LBs, LNs, and GCIs. Immunoelectron microscopic studies have demonstrated that these fibrils are intensely labeled with α-synuclein antibodies in situ. Sarcosyl-insoluble α-synuclein filaments with straight and twisted morphologies can also be observed in extracts of DLBD and MSA brains. Moreover, α-synuclein can assemble in vitro into elongated homopolymers with similar widths as sarcosyl-insoluble fibrils or filaments visualized in situ. Polymerization is associated with a concomitant change in secondary structure from random coil to anti-parallel β-sheet structure consistent with the Thioflavine-S reactivity of these filaments. Furthermore, the PD-association with α-synuclein mutation, A53T, may accelerate this process, as recombinant A53T α-synuclein has a greater propensity to polymerize than wild-type α-synuclein. This mutation also affects the ultrastructure of the polymers; the filaments are slightly wider and are more twisted in appearance, as if assembled from two protofilaments. The A30P mutation may also modestly increase the propensity of α-synuclein to polymerize, but the pathological effects of this mutation also may be related to its reduced binding to vesicles. Interestingly, carboxyl-terminally truncated α-synuclein may be more prone to form filaments than the full-length protein.

In certain embodiments, an FTI is used in accordance with the present invention to treat a subject with the synucleinopathy: Parkinson's disease. Parkinson's disease (PD) is a neurological disorder characterized by bradykinesia, rigidity, tremor, and postural instability, as well as other non-motor symptoms. The pathologic hallmarks of PD are the loss of neurons in the substantia nigra pars compacta (SNpc) and the appearance of Lewy bodies in remaining neurons. It appears that more than about 50% of the cells in the SNpc need to be lost before motor symptoms appear. Associated symptoms often include small handwriting (micrographia), seborrhea, orthostatic hypotension, urinary difficulties, constipation and other gastrointestinal dysfunction, sleep disorders, depression and other neuropsychiatric phenomena, dementia, and smelling disturbances (occurs early). Patients with Parkinsonism have greater mortality, about two times compared to general population without PD. This is attributed to greater frailty or reduced mobility.

Diagnosis of PD is mainly clinical and is based on the clinical findings listed above. Parkinsonism, refers to any combination of two of bradykinesia, rigidity, and/or tremor. PD is the most common cause of parkinsonism. Other causes of parkinsonism are side effects of drugs, mainly the major tranquilizers, such as Haldol, strokes involving the basal ganglia, and other neurodegenerative disorders, such as Diffuse Lewy Body Disease (DLBD), progressive supranuclear palsy (PSP), frontotemporal dementia (FTD), MSA, and Huntington's disease. The pathological hallmark of PD is the Lewy body, an intracytoplasmatic inclusion body typically seen in affected neurons of the substantia nigra and to a variable extent, in the cortex. Recently, α-synuclein has been identified as the main component of Lewy bodies in sporadic Parkinsonism.

Although parkinsonism can be clearly traced to viruses, stroke, or toxins in a few individuals, for the most part, the cause of Parkinson's disease in any particular case is unknown. Environmental influences which may contribute to PD may include drinking well water, farming and industrial exposure to heavy metals (e.g., iron, zinc, copper, mercury, magnesium and manganese), alkylated phosphates, and orthonal chlorines. Paraquat (a herbicide) has also been associated with increased prevalence of Parkinsonism including PD. Cigarette smoking is associated with a decreased incidence of PD. The current consensus is that PD may either be caused by an uncommon toxin combined with high genetic susceptibility or a common toxin combined with relatively low genetic susceptibility.

A small percentage of subjects that are at risk of developing PD can be identified for example by genetic analysis. There is good evidence for certain genetic factors being associated with PD. Large pedigrees of autosomal dominantly inherited PDs have been reported. For example, a mutation in α-synuclein is responsible for one pedigree and triplication of the SNCA gene (the gene coding for α-synuclein) is associated with PD in others.

According to the invention, the term synucleinopathic subject also encompasses a subject that is affected by, or is at risk of developing DLBD. FTIs in accordance with the present invention may be used to treat a subject with DLBD. These subjects can be readily identified by persons of ordinary skill in the art by symptomatic diagnosis or by genetic screening, brain scans, SPECT, PET imaging, etc.

DLBD is the second most common cause of neurodegenerative dementia in older people, it effects 7% of the general population older than 65 years and 30% of those aged over 80 years. It is part of a range of clinical presentations that share a neurotic pathology based on normal aggregation of the synaptic protein α-synuclein. DLBD has many of the clinical and pathological characteristics of the dementia that occurs during the course of Parkinson's disease. In addition to other clinical and neurologic diagnostic criteria, a “one year rule” can been used to separate DLBD from PD. According to this rule, onset of dementia within 12 months of Parkinsonism qualifies as DLBD, whereas more than 12 months of Parkinsonism before onset of dementia qualifies as PD. The central features of DLBD include progressive cognitive decline of sufficient magnitude to interfere with normal social and occupational function. Prominent or persistent memory impairment does not necessarily occur in the early stages, but it is evident with progression in most cases. Deficits on tests of attention and of frontal cortical skills and visual spatial ability can be especially prominent Core diagnostic features, two of which are essential for diagnosis of probable and one for possible DLBD are fluctuating cognition with pronounced variations in attention and alertness, recurrent visual hallucinations that are typically well-formed and detailed, and spontaneous features of Parkinsonism. In addition, there can be some supportive features, such as repeated falls, syncope, transient loss of consciousness, neuroleptic sensitivity, systematized delusions, hallucinations and other modalities, REM sleep behavior disorder, and depression. Patients with DLBD do better than those with Alzheimer's Disease in tests of verbal memory, but worse on visual performance tests. This profile can be maintained across the range of severity of the disease, but can be harder to recognize in the later stages owing to global difficulties. DLBD typically presents with recurring episodes of confusion on a background of progressive deterioration. Patients with DLBD show a combination of cortical and subcortical neuropsychological impairments with substantial attention deficits and prominent frontal subcortical and visual spatial dysfunction. These help differentiate this disorder from Alzheimer's disease.

Rapid eye movement (REM), sleep behavior disorder is a parasomnia manifested by vivid and frightening dreams associated with simple or complex motor behavior during REM sleep. This disorder is frequently associated with the synucleinopathies, DLBD, PD, and MSA, but it rarely occurs in amyloidopathies and taupathies. The neuropsychological pattern of impairment in REM sleep behavior disorder/dementia is similar to that reported in DLBD and qualitatively different from that reported in Alzheimer's disease. Neuropathological studies of REM sleep behavior disorder associated with neurodegenerative disorder have shown Lewy body disease or multiple system atrophy. REM sleep wakefulness disassociations (REM sleep behavior disorder, daytime hypersomnolence, hallucinations, cataplexy) characteristic of narcolepsy can explain several features of DLBD, as well as PD. Sleep disorders could contribute to the fluctuations typical of DLBD, and their treatment can improve fluctuations and quality of life. Subjects at risk of developing DLBD can be identified. Repeated falls, syncope, transient loss of consciousness, and depression are common in older people with cognitive impairment and can serve as (a red flag) to a possible diagnosis of DLBD. By contrast, narcoleptic sensitivity in REM sleep behavior disorder can be highly predictive of DLBD. Their detection depends on the clinicians having a high index of suspicion and asking appropriate screening questions.

Clinical diagnosis of synucleinopathic subjects that are affected by or at risk of developing LBD can be supported by neuroimaging investigations. Changes associated with DLBD include preservation of hippocampal, and medialtemporal lobe volume on MRI and occipital hypoperfusion on SPECT. Other features, such as generalized atrophy, white matter changes, and rates of progression of whole brain atrophy are not helpful in differential diagnosis. Dopamine transporter loss in the caudate and putamen, a marker of nigrostriatal degeneration, can be detected by dopamenergic SPECT and can prove helpful in clinical differential diagnosis. A sensitivity of 83% and specificity of 100% has been reported for an abnormal scan with an autopsy diagnosis of DLBD.

Consensus criteria for diagnosing DLBD include ubiquitin immunohistochemistry for Lewy body identification and staging into three categories; brain stem predominant, limbic, or neocortical, depending on the numbers and distribution of Lewy bodies. The recently-developed α-synuclein immunohistochemistry can visualize more Lewy bodies and is also better at indicating previously under recognized neurotic pathology, termed Lewy neurites. Use of antibodies to α-synuclein moves the diagnostic rating for many DLBD cases from brain stem and limbic groups into the neocortical group.

In most patients with DLBD, there are no genetic mutations in the α-synuclein or other Parkinson's disease-associated genes. Pathological up-regulation of normal, wild-type α-synuclein due to increased mRNA expression is a possible mechanism, or Lewy bodies may form because α-synuclein becomes insoluble or more able to aggregate. Another possibility is that α-synuclein is abnormally processed, for example, by a dysfunctional proteasome system and that toxic “proto fibrils” are therefore produced. Sequestering of these toxic fibrils into Lewy bodies could reflect an effort by the neurons to combat biological stress inside the cell, rather than their simply being neurodegenerative debris.

Target symptoms for the accurate diagnosis of DLBD can include extrapyramidal motor features, cognitive impairment, neuropsychiatric features (including hallucinations, depression, sleep disorder, and associated behavioral disturbances), or autonomic dysfunction.

Methods of the invention can be used in combination with one or more other medications for treating DLBD. For example, the lowest acceptable doses of levodopa can be used to treat DLBD. D2-receptor antagonists, particularly traditional neuroleptic agents, can provoke severe sensitivity reactions in DLBD subjects with an increase in mortality of two to three times. Cholinsterase inhibitors discussed above are also used in the treatment of DLBD.

In certain embodiments, FTIs are used in accordance with the present invention to treat multiple system atrophy. MSA is a neurodegenerative disease marked by a combination of symptoms; affecting movement, cognition, autonomic and other body functions, hence the label “multiple system atrophy”. The cause of MSA is unknown. Symptoms of MSA vary in distribution of onset and severity from person to person. Because of this, the nomenclature initially included three distinct terms: Shy-Drager syndrome, striatonigral degeneration (SD), and olivopontocerebellar atrophy (OPCA).

In Shy-Drager syndrome, the most prominent symptoms are those involving the autonomic system; blood pressure, urinary function, and other functions not involving conscious control. Striatonigral degeneration causes Parkinsonism symptoms, such as slowed movements and rigidity, while OPCA principally affects balance, coordination, and speech. The symptoms for MSA can also include orthostatic hypertension, male impotence, urinary difficulties, constipation, speech and swallowing difficulties, and blurred vision.

The initial diagnosis of MSA is usually made by carefully interviewing the patient and performing a physical examination. Several types of brain imaging, including computer tomography, scans, magnetic resonance imaging (MRI), and positron emission tomography (PET), can be used as corroborative studies. An incomplete and relatively poor response to dopamine replacement therapy, such as Sinemet, may be a clue that the presentation of bradykinesia and rigidity (parkinsonism) is not due to PD. A characteristic involvement of multiple brain systems with prominent autonomic dysfunction is a defining feature of MSA and one that at autopsy confirms the diagnosis. Patients with MSA can have the presence of glial cytoplasmic inclusions in certain types of brain cells, as well. Prototypic Lewy bodies are not present in MSA. However, α-synuclein staining by immunohistochemistry is prominent. In comparison to Parkinson's disease, in addition to the poor response to Sinemet, there are a few other observations that are strongly suggested for MSA, such as postural instability, low blood pressure on standing (orthostatic hypotension) and high blood pressure when lying down, urinary difficulties, impotence, constipation, speech and swallowing difficulties out of proportion to slowness and rigidity.

Methods of the invention can be used in combination with one or more alternative medications for treating MSA. Typically, the drugs that can be used to treat various symptoms of MSA become less effective as the disease progresses. Levodopa and dopamine agonists used to treat PD are sometimes effective for the slowness and rigidity of MSA. Orthostatic hypertension can be improved with cortisone, midodrine, or other drugs that raise blood pressure. Male impotence may be treated with penile implants or drugs. Incontinence may be treated with medication or catheterization. Constipation may improve with increased dietary fiber or laxatives.

### Amyloidopathy

The present invention provides methods relevant to amyloidopathies. For example, in some embodiments, the present invention provides a method of reducing amyloid beta toxicity in a cell, the method comprising administering to a cell a therapeutically effective amount of a provided compound. In some embodiments, the present invention provides a method of reducing the accumulation of amyloid beta proteins in a cell, the method comprising administering to a cell a therapeutically effective amount of a provided compound. In some embodiments, the cell is a neuronal cell. In some embodiments, the cell expresses amyloid beta proteins. In some embodiments, the present invention provides a method of reducing amyloid beta toxicity in the brain, the method comprising administering to a human a therapeutically effective amount of a provided compound. In some embodiments, the present invention provides a method of reducing the accumulation of amyloid beta proteins in the brain, the method comprising administering to a human a therapeutically effective amount of a provided compound. In certain embodiments, the amyloidopathy is Alzheimer's disease.

### Taupathy

The present invention provides methods related to taupathies. Taupathies are neurodegenerative disorders characterized by the presence of filamentous deposits, consisting of hyperphosphorylated tau protein, in neurons and glia. Abnormal tau phosphorylation and deposition in neurons and glial cells is one of the major features in taupathies. The term tauopathy, was first used to describe a family with frontotemporal dementia (PTD) and abundant tau deposits. This term is now used to identify a group of diseases with widespread tau pathology in which tau accumulation appears to be directly associated with pathogenesis. Major neurodegenerative taupathies includes sporadic and hereditary diseases characterized by filamentous tau deposits in brain and spinal cord.

In the majority of taupathies, glial and neuronal tau inclusions are the sole or predominant CNS lesions. Exemplary such taupathies include amyotrophic lateral sclerosis (ALS), parkinsonism, argyrophilic grain dementia, diffuse neurofibrillary tangles with calcification, frontotemporal dementia linked to chromosome 17, corticobasal degeneration, Pick's disease, progressive supranuclear palsy, progressive subcortical gliosis, and tangle only dementia.

Additionally, taupathies characterize a large group of diseases, disorders and conditions in which significant filaments and aggregates of tau protein are found. Exemplary such diseases, disorders, and conditions include sporadic and/or familial Alzheimer's Disease (AD), amyotrophic lateral sclerosis/parkinsonism-dementia complex (ALS-FTDP), argyrophilic grain dementia, dementia pugilistica, diffuse neurofibrillary tangles with calcification, Down syndrome, frontotemporal dementia, parkinsonism linked to chromosome 17 (FTDP-17), Gerstmann-Straussler-Scheinker disease, Hallervorden-Spatz disease, inclusion body myositis, Creutzfeld-Jakob disease (CJD), multiple system atrophy, Niemann-Pick disease (NPC), Pick's disease, prion protein cerebral amyloid angiopathy, progressive supranuclear palsy (PSP), subacute sclerosing panencephalitis, tangle-predominant Alzheimer's disease, corticobasal degeneration, (CBD), myotonic dystrophy, non-guanamian motor neuron disease with neurofibrillary tangles, postencephalitic parkinsonism, prion protein cerebral amyloid angiopathy, progressive subcortical gliosis, subacute sclerosing panencephalitis, and tangle-only dementia.

Neurodegenerative diseases where tau pathology is found in conjunction with other abnormal protein lesions may be considered secondary taupathies. Examples include Alzheimer's Disease (AD) and certain diseases where prion protein, Bri, or α-synuclein are aggregated. Although tau is probably not the initial pathological factor, tau aggregates contribute to the final degeneration.

### Cognitive Impairment

The present invention provides methods related to cognitive impairment. Cognitive impairment refers to a subject that is diagnosed with, affected by, or at risk of developing cognitive impairment or dementia. The cognitive impairment or dementia may stem from any etiology. Exemplary causes of cognitive impairment and dementia include neurodegenerative diseases, neurological diseases, psychiatric disorders, genetic diseases, infectious diseases, metabolic diseases, cardiovascular diseases, vascular diseases, aging, trauma, malnutrition, childhood diseases, chemotherapy, autoimmune diseases, and inflammatory diseases. Particular diseases that are associated with cognitive impairment or dementia include, but are not limited to, atherosclerosis, stroke, cerebrovascular disease, vascular dementia, multi-infarct dementia, Parkinson's disease and Parkinson's disease dementia, Lewy body disease, Pick's disease, Alzheimer's disease, mild cognitive impairment, Huntington's disease, AIDS and AIDS-related dementia, brain neoplasms, brain lesions, epilepsy, multiple sclerosis, Down's syndrome, Rett's syndrome, progressive supranuclear palsy, frontal lobe syndrome, schizophrenia, traumatic brain injury, post coronary artery by-pass graft surgery, cognitive impairment due to electroconvulsive shock therapy, cognitive impairment due to chemotherapy, cognitive impairment due to a history of drug abuse, attention deficit disorder (ADD), attention deficit hyperactivity disorder (ADHD), autism, dyslexia, depression, bipolar disorder, post-traumatic stress disorder, apathy, myasthenia gravis, cognitive impairment during waking hours due to sleep apnea, Tourette's syndrome, autoimmune vasculitis, systemic lupus erythematosus, polymyalgia rheumatica, hepatic conditions, metabolic diseases, Kufs' disease, adrenoleukodystrophy, metachromatic leukodystrophy, storage diseases, infectious vasculitis, syphillis, neurosyphillis, Lyme disease, complications from intracerebral hemorrhage, hypothyroidism, B12 deficiency, folic acid deficiency, niacin deficiency, thiamine deficiency, hydrocephalus, complications post anoxia, prion disease (Creutzfeldt-Jakob disease), Fragile X syndrome, phenylketonuria, malnutrition, neurofibromatosis, maple syrup urine disease, hypercalcemia, hypothyroidism, hypercalcemia, and hypoglycemia. The degree of cognitive impairment may be assessed by a health care professional. A variety of standardized test are available for assessing cognition, including, but not limited to, the Mini-Mental Status Examination, the Dementia Symptom Assessment Scale, and the ADAS. Such tests typically provide a measurable score of cognitive impairment. In certain embodiments, the cognitive impairment being treated or prevented is associated with Alzheimer's disease. In certain embodiments, the cognitive impairment is associated with a psychiatric disorder (e.g., schizophrenia). In certain embodiments, the cognitive impairment being treated or prevented is associated with a genetic disease. In certain embodiments, the cognitive impairment being treated or prevented is associated with an infectious disease (e.g., HIV, syphillis).

Dementia is commonly defined as a progressive decline in cognitive function due to damage or disease in the body beyond what is expected from normal aging. Dementia is described as a loss of mental function, involving problems with memory, reasoning, attention, language, and problem solving. Higher level functions are typically affected first. Dementia interferes with a person's ability to function in normal daily life. The present invention includes a method of treating vascular dementia.

### Depression

The present invention provides methods related to depression. Depression refers to a subject that is diagnosed with, affected by, or at risk of developing depression. Based on the treatment of a transgenic mouse overexpressing Tau with a farnesyl transferase inhibitor, reduced Tau transgene-induced depression was seen in the treated mice indicated by an increase in struggling and decreased floating in the forced swim test as compared to control animals. In addition, FTI-treated mice overexpressing TAU displayed behavior similar to non-transgenic animals. The treated mice also showed reduced phosphorylated TAU in the amygdala.

### Anxiety

The present invention provides methods related to anxiety. Anxiety refers to a subject that is diagnosed with, affected by, or at risk of developing a state of apprehension and psychic tension occurring in some forms of mental disorder/s. The anxiety state may stern from a variety of causes. Based on mouse studies, farnesyl transferase inhibitors may be used as anxiolytics.

### Lysosomal Storage Diseases

The present invention provides methods related to lysosomal storage disease. Lysosomal Storage diseases can result from a number of defects, including a primary defect in a lysosomal enzyme's activity, e.g. as in Gaucher disease or Fabry disease, or a defect the post-translational processing of a lysosomal enzyme eg as in Mucosuphatidosis, or a defect in the trafficking of a lysosomal enzyme eg as in Mucolipidosis type IIIA, or a defect in a lysosomal protein that is not an enzyme eg as in Danon disease, or a defect in a non-lysosomal protein eg as in a variant of Late Infantile Neuronal Ceroid Lipofuscinosis. In Lysosomal Storage disorders, there is often an accumulation of certain lipids e.g. glucosylceramide or cholesterol, or of certain proteins eg subunit c of ATP synthase, or of certain damaged organelles or organelle fragments e.g. fragmented mitochondria. Drug-induced stimulation of a cellular phagic response may be of therapeutic benefit in Lysosomal Storage disorders; such phagic responses may include microautophagy, macroautophagy, chaperone-mediated autophagy, mitophagy, pexophagy.

Representative lysosomal storage diseases include, for example, Activator Deficiency/GM2 Gangliosidosis, Alpha-mannosidosis, Aspartylglucosaminuria, beta-mannosidosis, carbohydrate-deficient glycoprotein syndrome, Cholesteryl ester storage disease, Chronic Hexosaminidase A Deficiency, cobalamin deficiency type F, Cystinosis, Damon disease, Fabry disease, Farber disease, Fucosidosis, Galactosialidosis, Gaucher Disease (e.g., Type I, Type II, Type III), GM1 gangliosidosis (e.g., Infantile, Late infantile/Juvenile, Adult/Chronic), GM₁ gangliosidosis, GM₂ gangliosidosis, GM₃; gangliosidosis, glycogen storage disease type II, I-Cell disease/Mucolipidosis IT, Infantile Free Sialic Acid Storage Disease/ISSD, Juvenile Hexosaminidase A Deficiency, Kanzaki disease, Krabbe disease (e.g., Infantile Onset, Late Onset), lactosylceramidosis, Metachromatic Leukodystrophy, Mucopolysaccharidoses disorders, Pseudo-Hurler polydystrophy/Mucolipidosis IIIA (e.g., MPSI Hurler Syndrome, MPSI Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS 1X Hyaluronidase Deficiency, MPS VI Maroteaux-Lamy, MPS VII Sly Syndrome, Mucolipidosis I/Sialidosis, Mucolipidosis IIIC, Mucolipidosis type IV), Multiple sulfatase deficiency, Niemann-Pick Disease (e.g., Type A, Type B, Type C), Neuronal Ceroid Lipofuscinoses (e.g., CLN6 disease—Atypical Late Infantile, Late Onset variant, Early Juvenile, Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis), Pompe disease/Glycogen storage disease type II, Pompe disease, Pycnodysostosis, Sandhoff disease/GM2 Gangliosidosis (e.g., Adult Onset, Infantile, Juvenile), Schindler disease, Salla disease/Sialic Acid Storage Disease, sialic acid storage disease, sialidosis, Tay-Sachs/GM2 gangliosidosis, or Wolman disease.

### Immunologic Disease

The present invention provides methods related to an immune disease or disorder. Immune diseases or disorders are for example, rejection following transplantation of synthetic or organic grafting materials, cells, organs or tissue to replace all or part of the function of tissues, such as heart, kidney, liver, bone marrow, skin, cornea, vessels, lung, pancreas, intestine, limb, muscle, nerve tissue, duodenum, small-bowel, pancreatic-islet-cell, including xenotransplants, etc. The invention further may be related to treatment of immune disease including treatment or preventing of graft-versus-host disease, autoimmune diseases, such as rheumatoid arthritis, systemic lupus erythematosus, thyroiditis, Hashimoto's thyroiditis, multiple sclerosis, myasthenia gravis, type I diabetes uveitis, juvenile-onset or recent-onset diabetes mellitus, uveitis, Graves' disease, psoriasis, atopic dermatitis, Crohn's disease, ulcerative colitis, vasculitis, auto-antibody mediated diseases, aplastic anemia, Evan's syndrome, autoimmune hemolytic anemia, and the like. The invention further relates to treatment or prevention of infectious diseases causing aberrant immune response and/or activation, such as traumatic or pathogen induced immune dysregulation, including for example, that which are caused by hepatitis B and C infections, HIV, Staphylococcus aureus infection, viral encephalitis, sepsis, parasitic diseases wherein damage is induced by an inflammatory response (e.g., leprosy).

In some embodiments, the invention relates to treatment or prevention of graft vs host disease (especially with allogenic cells), rheumatoid arthritis, systemic lupus erythematosus, psoriasis, atopic dermatitis, Crohn's disease, ulcerative colitis, other forms of inflammatory bowel disease (collagenous colitis, lymphocytic colitis, ischemic colitis, diversion colitis, Behcet's syndrome, infective colitis, indeterminate colitis) and/or multiple sclerosis.

Alternatively or additionally, in some embodiments, the invention relates to treatment or prevention of an immune response associated with a gene therapy treatment, such as the introduction of foreign genes into autologous cells and expression of the encoded product.

Exemplary of diseases caused or worsened by the host's own immune response are autoimmune diseases such as multiple sclerosis, lupus erythematosus, psoriasis, pulmonary fibrosis, and rheumatoid arthritis and diseases in which the immune response contributes to pathogenesis such as atherosclerosis, inflammatory diseases, osteomyelitis, ulcerative colitis, Crohn's disease, and graft versus host disease (GVHD) often resulting in organ transplant rejection. Additional exemplary inflammatory disease states include fibromyalgia, osteoarthritis, sarcoidosis, systemic sclerosis, Sjogren's syndrome, inflammations of the skin (e.g., psoriasis), glomerulonephritis, proliferative retinopathy, restenosis, and chronic inflammations.

### Mitochondrial Disease

The present invention provides methods related to mitochondrial disease. Mitochondrial diseases may be caused by mutations, acquired or inherited, in mitochondrial DNA or in nuclear genes that code for mitochondrial components. They may also be the result of acquired mitochondrial dysfunction due to adverse effects of drugs, infections, or other environmental causes.

Mitochondrial DNA inheritance behaves differently from autosomal and sex-linked inheritance. Mitochondrial DNA, unlike nuclear DNA, is strictly inherited from the mother and each mitochondrial organelle typically contains multiple mtDNA copies. During cell division, the mitochondrial DNA copies segregate randomly between the two new mitochondria, and then those new mitochondria make more copies. As a result, if only a few of the mtDNA copies inherited from the mother are defective, mitochondrial division may cause most of the defective copies to end up in just one of the new mitochondria. Mitochondrial disease may become clinically apparent once the number of affected mitochondria reaches a certain level; this phenomenon is called ‘threshold expression’. Mitochondrial DNA mutations occur frequently, due to the lack of the error checking capability that nuclear DNA has. This means that mitochondrial DNA disorders may occur spontaneously and relatively often. In addition, defects in enzymes that control mitochondrial DNA replication may cause mitochondrial DNA mutations.

Mitochondrial diseases include any clinically heterogeneous multisystem disease characterized by mutations of the brain-mitochondrial encephalopathies and/or muscle-mitochondrial myopathies due to alterations in the protein complexes of the electron transport chain of oxidative phosphorylation. In some embodiment, the invention relates to the treatment or prevention of a mitochondrial diseases. For example, the invention provides methods for the treatment or prevention of Leber's hereditary optic atrophy, MERRF (Myoclonus Epilepsy with Ragged Red Fibers), MELAS (Mitochondrial Encephalopathy, Lactic Acidosis and Stroke-like episodes); Alper syndrome, Lowe syndrome, Luft syndrome, Menke's kinky hair syndrome, Zellweger syndrome, mitochondrial myopathy, and rhizomelic chondrodysplasia punctata.

While not intending to be bound to any particular theory, compounds of the invention protect against neuronal dysfunction and death that causes the neurologic symptoms (e.g., cognitive losses, muscle weakness, cardiac dysfunction) diseases that are characterized by mitochondrial dysfunction. In these diseases, dysfunctional mitochondria accumulate. The normal mechanism of mitochondria recycling is unable to keep up with the increased demand. Compounds of the invention stimulate the so-called mitophagy pathway, leading to regeneration of fully functional mitochondria.

MELAS, MERFF, LHON (leber hereditary optic neuropathy), CPEO (chronic progressive external opthalmoplegia), KSS (Kearns-Sayre syndrome), MNGIE (mitochondrial neurogastrointestinal encephalopathy), NARP (neuropathy, ataxia, retinitis pigmentosa and ptosis), Leigh syndrome, Alpers-Huttenlocher disease, Kearns-Sayre syndrome, Pearson syndrome, and Luft disease are examples of mitochondrial diseases treatable by this mechanism.

### Ocular Disease

The present invention provides methods related to ocular disease. In some embodiments, compounds of the invention are useful for the treatment of ocular indications that benefit from a compound that simulates cellular autophagy. Ocular indications include but are not limited to retinitis pigmentosa, wet and dry forms of age related macular degeneration, ocular hypertension, glaucoma, corneal dystrophies, retinoschises, Stargardt's disease, autosomal dominant druzen, Best's macular dystrophy, myocilin glaucoma, or Malattia Leventineses.

### Inflammatory Disease

The present invention provides methods related to inflammatory disease. In certain embodiments, inflammatory diseases, disorders, and conditions may include one or more of inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendictitis, pancreatitis, cholocystitus, irritable bowel syndrome, ulcerative colitis, glomerulonephritis, dermatomyositis, scleroderma, vasculitis, allergic disorders including asthma such as bronchial, allergic, intrinsic, extrinsic and dust asthma, particularly chronic or inveterate asthma (e.g. late asthma airways hyper-responsiveness) and bronchitis, chronic obstructive pulmonary disease (COPD), multiple sclerosis, rheumatoid arthritis, disorders of the gastrointestinal tract, including, without limitation, Coeliac disease, proctitis, eosinophilic gastro-enteritis, mastocytosis, pancreatitis, Crohn's disease, ulcerative colitis, food-related allergies which have effects remote from the gut, e.g. migraine, rhinitis and eczema. Conditions characterised by inflammation of the nasal mucus membrane, including acute rhinitis, allergic, atrophic thinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofulous rhinitis, seasonal rhinitis including rhinitis nervosa (hay fever) and vasomotor rhinitis, sarcoidosis, farmer's lung and related diseases, fibroid lung and idiopathic interstitial pneumonia, acute pancreatitis, chronic pancreatitis, and adult respiratory distress syndrome, and/or acute inflammatory responses (such as acute respiratory distress syndrome and ischemia/reperfusion injury).

### Cardiovascular Disease

The present invention provides methods related to cardiovascular disease. Exemplary particular cardiovascular diseases, disorders and conditions may include one or more of myocardial ischemia, myocardial infarction, vascular hyperplasia, cardiac hypertrophy, congestive heart failure, cardiomegaly, restenosis, atherosclerosis, hypertension, and/or angina pectoris. In certain embodiments, the cardiovascular disease, disorder or condition is atherosclerosis, a coronary heart disease, an acute coronary symptom, unstable angina pectoris or acute myocardial infarction, stable angina pectoris, stroke, ischemic stroke, inflammation or autoimmune disease associated atherosclerosis or restenosis. In some embodiments, the invention relates to treatment or prevention of circulatory diseases, such as arteriosclerosis, atherosclerosis, vasculitis, polyarteritis nodosa and/or myocarditis.

### Proliferative Disease

The present invention provides methods related to proliferative disease. In general, cell proliferative disorders, diseases or conditions encompass a variety of conditions characterized by aberrant cell growth, preferably abnormally increased cellular proliferation. For example, cell proliferative disorders, diseases, or conditions include, but are not limited to, cancer, immune-mediated responses and diseases (e.g., transplant rejection, graft vs host disease, immune reaction to gene therapy, autoimmune diseases, pathogen-induced immune dysregulation, etc.), certain circulatory diseases, and certain neurodegenerative diseases.

In certain embodiments, the invention relates to methods of treating or preventing cancer. In general, cancer is a group of diseases which are characterized by uncontrolled growth and spread of abnormal cells. Examples of such diseases are carcinomas, sarcomas, leukemias, lymphomas and the like.

For example, cancers include, but are not limited to leukemias and lymphomas such as cutaneous T-cell lymphomas (CTCL), peripheral T-cell lymphomas, lymphomas associated with human T-cell lymphotrophic virus (HTLV) such as adult T-cell leukemia/lymphoma (ATLL), B-cell lymphoma, acute lymphocytic leukemia, acute nonlymphocytic leukemias, chronic lymphocytic leukemia, chronic myelogenous leukemia, acute myelogenous leukemia, Hodgkin's disease, non-Hodgkin's lymphomas, multiple myeloma, myelodysplastic syndrome, mesothelioma, common solid tumors of adults such as head and neck cancers (e.g., oral, laryngeal and esophageal), genitourinary cancers (e.g., prostate, bladder, renal, uterine, ovarian, testicular, rectal and colon), lung cancer, breast cancer, pancreatic cancer, melanoma and other skin cancers, stomach cancer, brain tumors, liver cancer and thyroid cancer, and/or childhood solid tumors such as brain tumors, neuroblastoma, retinoblastoma, Wilms' tumor, bone tumors, and soft-tissue sarcomas.

In some embodiments, the invention relates to treatment or prevention of leukemias. For example, in some embodiments, the invention relates to treatment or prevention of chronic lymphocytic leukemia, chronic myelogenous leukemia, acute lymphocytic leukemia, acute myelogenous leukemia, and/or adult T cell leukemia/lymphoma. In certain embodiments, the invention relates to the treatment or prevention of AML. In certain embodiments, the invention relates to the treatment or prevention of ALL. In certain embodiments, the invention relates to the treatment or prevention of CML. In certain embodiments, the invention relates to the treatment or preventing of CLL.

In some embodiments, the invention relates to treatment or preventing of lymphomas. For example, in some embodiments, the invention relates to treatment or prevention of Hodgkin's or non-Hodgkin's (e.g., T-cell lymphomas such as peripheral T-cell lymphomas, cutaneous T-cell lymphomas, etc.) lymphoma.

In some embodiments, the invention relates to the treatment or prevention of myelomas and/or myelodysplastic syndromes. In some embodiments, the invention relates to treatment or prevention of solid tumors. In some such embodiments the invention relates to treatment or prevention of solid tumors such as lung, breast, colon, liver, pancreas, renal, prostate, ovarian, and/or brain. In some embodiments, the invention relates to treatment or prevention of pancreatic cancer. In some embodiments, the invention relates to treatment or prevention of renal cancer. In some embodiments, the invention relates to treatment or prevention of prostate cancer. In some embodiments, the invention relates to treatment or prevention of sarcomas. In some embodiments, the invention relates to treatment or prevention of soft tissue sarcomas. In some embodiments, the invention relates to methods of treating or preventing one or more immune-mediated responses and diseases.

Without wishing to be bound by a particular theory, inhibition of the farnesylation of UCH-L I or another non-CaaX-CO₂H FTase substrate is thought to stimulate autophagy, thereby increasing protein clearance. Inhibition of the farnesylation of UCH-L1 or another non-CaaX—CO₂H-FTase substrate can be achieved at lower doses of an FTI than are needed to inhibit the farnesylation of Ras protein. Therefore, doses of FTIs useful in the treatment of proteinopathies, as compared to cancer, are lower. In certain embodiments, the dosing of an FTI in the treatment of a proteinopathy is approximately 2-fold, 5-fold, 10-fold, 20-fold, 25-fold, 50-fold, 100-fold, 500-fold, or 1000-fold less than the equivalent dosing in humans of therapeutically effective doses observed in xenograft models of cancer.

In some embodiments, an FTI or pharmaceutical composition of the invention is provided to a subject with a proteinopathy chronically. Chronic treatments include any form of repeated administration for an extended period of time, such as repeated administrations for one or more months, between a month and a year, one or more years, or longer. In many embodiments, a chronic treatment involves administering an FTI or pharmaceutical composition thereof repeatedly over the life of the subject. Preferred chronic treatments involve regular administrations, for example one or more times a day, one or more times a week, or one or more times a month. In certain embodiments, the treatment is intermittent. Preferred intermittent treatments would involve dosing every other day, every third day, etc. An alternative intermittent treatment would involve dosing every day for a period of time followed by cessation of dosing for an equal or greater amount of time. For example, the treatment may involve three days on followed by three day off; five days on followed by five days off, 7 days on followed by 7 days off, and so on. Such intermittent treatment may be continued long term.

In general, a suitable dose such as a daily dose of an FTI will be that amount of the FTI that is the lowest dose effective to produce a therapeutic effect. Such an effective dose will generally depend upon the factors described above.

In certain particular embodiments, for an adult human, the daily dose of the FTI (LNK-754 or Zarnestra® or pharmaceutically acceptable salt thereof) ranges from approximately 0.1 mg to 150 mg. In certain embodiments, the daily dosage ranges from approximately 0.1 mg to approximately 50 mg. In certain embodiments, the daily dose ranges from approximately 0.5 mg to approximately 30 mg. In certain embodiments, the daily dose ranges from approximately 4 mg to approximately 20 mg. In certain embodiments, the daily dose ranges from approximately 10 mg to approximately 30 mg. In certain embodiments, the daily dose ranges from approximately 10 mg to approximately 25 mg. In certain embodiments, the daily dose ranges from approximately 10 mg to approximately 30 mg. In certain embodiments, the daily dose of the FTI is approximately 1 mg, approximately 5 mg, approximately 10 mg, approximately 15 trig, approximately 20 mg, approximately 25 mg, approximately 30 mg, approximately 35 mg, approximately 40 mg, approximately 45 mg, or approximately 50 mg.

Generally doses of the FTI for a patient, when used for the indicated effects, will range from about 7 to 10,500 mg per kg of body weight per day. Preferably, the daily dosage will range from about 7 to 3500 mg per kg of body weight per day. More preferably the daily dosage will range from 35 to 2100 mg of compound per kg of body weight, and even more preferably from 280 to 1400 mg of compound per kg of body weight. However, lower or higher doses may be used. Such doses may correspond to doses found useful and appropriate in an applicable animal model (e.g., in a transgenic rodent model). In some embodiments, the dose administered to a subject may be modified as the physiology of the subject changes due to age, disease progression, weight, or other factors.

In certain embodiments, the area under the curve (AUC) resulting from the dosage of the FTI is less than approximately 2000 ng·hr/mL. In certain embodiments, the AUC is less than approximately 1500 ng·hr/mL. In certain embodiments, the AUC is less than approximately 1000 ng·hr/mL. In certain embodiments, the AUC is less than approximately 500 ng·hr/mL. In certain embodiments, the AUC is less than approximately 100 ng·hr/mL. In certain embodiments, the AUC is less than approximately 50 ng·hr/mL. In certain embodiments, the FTI is not administered every day but every other day, every third day, every fourth day, every other week, two weeks in a month, or every other month. In certain embodiments, the FTI is administered every other week. In certain embodiments, the FTI is administered every third week. In certain embodiments, the FTI is administered every fourth week. When the FTI is not administered for multiple days between doses, the dosing may be continued for a single day or multiple days. For example, when the FTI is administered every fourth week, it may be administered every day for a week followed by three weeks with no administration of the FTI. In certain embodiments, a controlled release formulation of the FTI is used to provide the desired daily dose as described above. In certain embodiments, the FTI is dosed intermittently. For example, the subject may be treated daily for a month and then the treatment may be stopped for 2-6 months, and then repeated.

Methods of the invention can be used in combination with one or more other medications, including medications that are currently used to treat proteinopathies arising as side-effects of the disease or of the aforementioned medications.

For example, methods of the invention can be used in combination with other pharmaceutical agents for treating PD. Levodopa mainly in the form of combination products containing carbodopa and levodopa (Sinemet and Sinemet CR) is the mainstay of treatment and is the most effective agent for the treatment of PD. Levodopa is a dopamine precursor, a substance that is converted into dopamine by an enzyme in the brain. Carbodopa is a peripheral decarboxylase inhibitor which prevents side effects and lower the overall dosage requirement. The starting dose of Sinemet is a 25/100 or 50/200 tablet prior to each meal. Dyskinesias may result from overdose and also are commonly seen after prolonged (e.g., years) use. Direct acting dopamine agonists may have less of this side effect. About 15% of patients do not respond to levodopa. Stalevo (carbodopa, levodopa, and entacapone) is a new combination formulation for patients who experience signs and symptoms of “wearing-off.” The formulation combines carbodopa and levodopa (the most widely used agents to treat AD) with entacapone, a catechol-O-methyltransferase inhibitor. While carbodopa reduces the side effects of levodopa, entacapone extends the time levodopa is active in the brain, up to about 10% longer.

Amantidine (SYMMETREL®) is a mild agent thought to work by multiple mechanisms including blocking the re-uptake of dopamine into presynaptic neurons. It also activates the release of dopamine from storage sites and has a glutamate receptor blocking activity. It is used as early monotherapy, and the dosing is 200 to 300 mg daily. Amantadine may be particularly helpful in patients with predominant tremor. Side effects include ankle swelling and red blotches. It may also be useful in later stage disease to decrease the intensity of drug-induced dyskinesia.

Anticholinergics (trihexyphenidyl, benztropine mesylate, procyclidine, artane, cogentin) do not act directly on the dopaminergic system. Direct-acting dopamine agonists include bromocriptidine (Parlodel), pergolide (Permax), ropinirol (Requip), and pramipexole (Mirapex). These agents cost substantially more than levodopa (Sinemet), and additional benefits are controversial. Depending on which dopamine receptor is being stimulated, D1 and D2 agonist can exert anti-Parkinson effects by stimulating the D1 and D2 receptors, such as Ergolide. Mirapex and Requip are the newer agents. Both are somewhat selected for dopamine receptors with highest affinity for the D2 receptor and also activity at the D3 receptor. Direct dopamine agonists, in general, are more likely to produce adverse neuropsychiatric side effects such as confusion than levodopa. Unlike levodopa, direct dopamine agonists do not undergo conversion to dopamine and thus do not produce potentially toxic free radical as they are metabolized. It is also possible that the early use of direct dopamine agonist decreases the propensity to develop the late complications associated with direct stimulation of the dopamine receptor by dopamine itself, such as the “on-off” effect and dyskinesia.

Monoaminoxidase-B inhibitors (MAO) such as selegiline (Deprenyl, or Eldepryl), taken in a low dose, may reduce the progression of Parkinsonism. These compounds can be used as an adjunctive medication. A study has documented that selegiline delays the need for levodopa by roughly three months, although interpretation of this data is confounded by the mild symptomatic benefit of the drug. Nonetheless, theoretical and in vitro support for a neuroprotective effect for some members of the selective MAOB class of inhibitors remains (e.g., rasagiline).

Catechol-O-methyltransferase inhibitors (COMT) can also be used in combination treatments of the invention. Catechol-O-methyltransferase is an enzyme that degrades levodopa, and inhibitors can be used to reduce the rate of degradation. Entacapone is a peripherally acting COMT inhibitor, which can be used in certain methods and compositions of the invention. Tamar or Tolcapone, approved by the FDA in 1997, can also be used in certain methods and compositions of the invention. Psychiatric adverse effects that are induced or exacerbated by PD medication include psychosis, confusion, agitation, hallucinations, and delusions. These can be treated by decreasing dopamine medication, reducing or discontinuing anticholinergics, amantadine or selegiline or by using low doses of atypical antipsychotics such as clozapine or quetiapine.

Methods of the invention can also be used in combination with surgical therapies for the treatment of PD. Surgical treatment is presently recommended for those who have failed medical management of PD. Unilateral thallamotomy can be used to reduce tremor. It is occasionally considered for patients with unilateral tremor not responding to medication. Bilateral procedures are not advised. Unilateral deep brain stimulation of the thalamus for tremor may also be a benefit for tremor. Unilateral pallidotomy is an effective technique for reducing contralateral drug-induced dyskinesias. Gamma knife surgery—thalamotomy or pallidotomy—can be performed as a radiological alternative to conventional surgery. The currently preferred neurosurgical intervention is, however, bilateral subthalamic nucleus stimulation. Neurotransplantation strategies remain experimental. In addition to surgery and medication, physical therapy in Parkinsonism maintains muscle tone, flexibility, and improves posture and gait.

The invention provides methods for treating a subject with a proteinopathy, comprising administering to a proteinopathic subject LNK-754 or Zarnestra® or a pharmaceutically acceptable salt thereof, in a therapeutically effective amount. In certain embodiments, the therapeutically effective amount is that amount needed to induce toxic protein clearance. In certain embodiments, the therapeutically effective amount is that amount needed to induce toxic protein clearance without substantially inhibiting the farnesylation of Ras. In certain embodiments, the therapeutically effective amount is that amount needed to inhibit the farnesylation of non-CaaX—CO₂H FTase substrates e.g., UCH-L1. In certain embodiments, the therapeutically effective amount is that amount needed to inhibit the farnesylation of a non-CaaX—CO₂H FTase substrates e.g., UCH-L1 without inhibiting the farnesylation of Ras to the extent necessary for the treatment of cancer. In certain embodiments, the therapeutically effective amount is the amount that leads to a 2-fold greater inhibition of the farnesylation of a non-CaaX—CO₂H FTase substrates e.g., UCH-L1 compared to the inhibition of the farnesylation of Ras. In certain embodiments, the therapeutically effective amount is the amount that leads to a 3-fold greater inhibition of the farnesylation of a non-CaaX—CO₂H FTase substrates e.g., UCH-L1 compared to the inhibition of the farnesylation of Ras. In certain embodiments, the therapeutically effective amount is the amount that leads to a 5-fold greater inhibition of the farnesylation of a non-CaaX—CO₂H FTase substrates e.g., UCH-L1 compared to the inhibition of the farnesylation of Ras. In certain embodiments, the therapeutically effective amount is the amount that leads to a 10-fold greater inhibition of the farnesylation of a non-CaaX—CO₂H FTase substrates e.g., UCH-L1 compared to the inhibition of the farnesylation of Ras. In certain embodiments, the therapeutically effective amount is the amount that leads to a 50-fold greater inhibition of the farnesylation of UCH-L1 compared to the inhibition of the farnesylation of Ras. In certain embodiments, the therapeutically effective amount is the amount that leads to a 100-fold greater inhibition of the farnesylation of a non-CaaX—CO₂H FTase substrates e.g., UCH-L1 compared to the inhibition of the farnesylation of Ras. In certain embodiments, the therapeutically effective amount is the amount that leads to a 500-fold greater inhibition of the farnesylation of a non-CaaX—CO₂H FTase substrates e.g., UCH-L1 compared to the inhibition of the farnesylation of Ras. In certain embodiments, the therapeutically effective amount is the amount that leads to a 1000-fold greater inhibition of the farnesylation of a non-CaaX—CO₂H FTase substrates e.g., UCH-L1 compared to the inhibition of the farnesylation of Ras. In some embodiments, the methods further comprise administering to the subject an amount of one or more non-farnesyl transferase inhibitor compounds effective to treat a neurological disorder. In some embodiments, the non-farnesyl transferase inhibitor compound is selected from the group consisting of dopamine agonist, DOPA decarboxylase inhibitor, dopamine precursor, monoamine oxidase blocker, cathechol O-methyl transferase inhibitor, anticholinergic, gamma-secretase inhibitor, PDE10 inhibitor, and NMDA antagonist. In some embodiments, the non-farnesyl transferase inhibitor is Memantine. In some embodiments, the non-farnesyl tranferase inhibitor compound is selected from the group consisting of Aricept and other acetylcholinesterase inhibitors.

The invention provides methods for treating proteinopathic disorders using farnesyl transferase inhibitors. It has been now discovered that UCH-L1 is farnesylated in vivo. UCH-L1 is associated with the membrane and this membrane association is mediated by farnesylation. Farnesylated UCH-L1 also stabilizes the accumulation of α-synuclein. In certain embodiments, the invention relates to the prevention or inhibition of UCH-L1 farnesylation which would result in UCH-L1 membrane disassociation and acceleration of the degradation of α-synuclein. Since α-synuclein accumulation is pathogenic in PD, DLBD, and MSA, an increased degradation of α-synuclein and/or inhibition of α-synuclein accumulation ameliorates the toxicity associated with a pathogenic accumulation of α-synuclein. In some embodiments, the invention provides methods of reducing α-synuclein toxicity in a cell, the method comprising administering to a cell a therapeutically effective amount of an inventive compound. In some embodiments, the cell is a neuronal cell. In some embodiments, the cell expresses α-synuclein.

The invention also provides methods for treating a proteinopathy using inhibitors of farnesyl transferase. Without wishing to be bound by a particular theory, the farnesyl transferase inhibitor is thought to activate autophagy. Another autophagy activator, rapamycin, has also been shown to have an anti-depressive effect in rodents. Cleary at a., Brain Research Bulletin 76:469-73, 2008.

The modification of a protein by a farnesyl group can have an important effect on function for a number of proteins. Farnesylated proteins typically undergo further C-terminal modification events that include a proteolytic removal of three C-terminal amino acids and carboxymethylation of C-terminal cysteines on their α-carbon carboxylate. These C-terminal modifications facilitate protein-membrane association as well as protein-protein interactions. Farnesylation is catalyzed by a protein farnesyltransferase (FTase), a heterodimeric enzyme that recognizes the CaaX motif present at the C-terminus of the substrate protein. The FTase transfers a farnesyl group from farnesyl pyrophosphate and forms a thioether linkage between the farnesyl and the cystine residues in the CaaX motif. A number of inhibitors of FTase have been developed and are known in the art.

### Pharmaceutical Compositions

The present invention also provides pharmaceutical compositions, preparations, and articles of manufacture comprising an FTI and a pharmaceutically acceptable carrier or excipient for use in accordance with the present invention. In some embodiments, the pharmaceutical composition, preparation, or article of manufacture further comprises one or more non-farnesyl transferase inhibitor compounds effective to treat a neurological disorder as described herein. Exemplary non-farnesyl transferase inhibitors are described herein.

The compositions, preparation, and articles of manufacture typically include amounts of each agent appropriate for the administration to a subject. In some embodiments, the article of manufacture comprises packaging material and an inventive compound. In some embodiments, the article of manufacture comprises a label or package insert indicating that the compound can be administered to a subject for treating a proteinopathy as described herein.

Pharmaceutical compositions of the present invention include those suitable for oral, nasal, topical (including buccal and sublingual), rectal, vaginal and/or parenteral administration. The compositions may conveniently be presented in unit dosage form and may be prepared by any methods well known in the art of pharmacy. The amount of active ingredient (i.e., farnesyl transferase inhibitor) which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

Methods of preparing these compositions include the step of bringing into association a farnesyl transferase inhibitor with the carrier and, optionally, one or more accessory ingredients. In general, the formulations are prepared by uniformly and intimately bringing into association an FTI with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. An FTI may also be administered as a bolus, electuary, or paste.

In solid dosage forms of the invention for oral administration (capsules, tablets, pills, dragees, powders, granules and the like), the active ingredient (i.e., farnesyl transferase inhibitor) is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically-acceptable carrier, and with any preservatives, buffers, or propellants which may be required.

The ointments, pastes, creams, and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an FTI, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an FTI to the body. Dissolving or dispersing the FTI in the proper medium can make such dosage forms. Absorption enhancers can also be used to increase the flux of the FTI across the skin. Either providing a rate controlling membrane or dispersing the FTI in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise an FTI in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms upon the FTI may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Examples of pharmaceutically acceptable antioxidants include water soluble antioxidants, such as ascorbic acid, cysteine hydrochloride, sodium bisulfate, sodium metabisulfite, sodium sulfite, and the like; oil-soluble antioxidants, such as ascorbyl palmitate, butylated hydroxyanisole (BHA), butylated hydroxytoluene (BHT), lecithin, propyl gal late, alpha-tocopherol, and the like; and metal chelating agents, such as citric acid, ethylenediamine tetraacetic acid (EDTA), sorbitol, tartaric acid, phosphoric acid, and the like.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a compound or pharmaceutical preparation is administered orally. In other embodiments, the compound or pharmaceutical preparation is administered intravenously. Alternative routes of administration include sublingual, intramuscular, and transdermal administrations.

When the FTIs are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compositions of the present invention may be given orally, parenterally, topically, or rectally. They are of course given in forms suitable for the administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the FTI, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt, or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In some embodiments, an FTI or pharmaceutical composition of the invention is provided to a proteinopathic subject. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the subject. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.1 mg to about 150 mg per day for an adult human subject. Preferably, the daily dosage will range from about 0.1 mg to about 50 mg per day for an adult human subject. More preferably, the daily dosage will range from about 0.5 mg to about 30 mg of compound per day, and even more preferably from about 4 mg to about 20 mg of compound per day. However, lower or higher doses can be used. In some embodiment, the effective daily dose of the active compound is administered once daily. If desired, the effective daily dose of the active compound may be administered as two, three, four, five, six or more sub-doses administered separately at appropriate intervals throughout the day, optionally, in unit dosage forms.

While it is possible for an FTI to be administered alone, it is preferable to administer the compound as a pharmaceutical formulation (composition) as described above.

The FTI may be formulated for administration in any convenient way for use in human or veterinary medicine, by analogy with other pharmaceuticals.

According to the invention, compounds for treating neurological conditions or diseases can be formulated or administered using methods that help the compounds cross the blood-brain barrier (BBB). The vertebrate brain (and CNS) has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood.

The unique morphologic characteristics of the brain capillaries that make up the BBB are (a) epithelial-like high resistance tight junctions which literally cement all endothelia of brain capillaries together, and (b) scanty pinocytosis or transendothelial channels, which are abundant in endothelia of peripheral organs. Due to the unique characteristics of the blood-brain barrier, hydrophilic drugs and peptides that readily gain access to other tissues in the body are barred from entry into the brain or their rates of entry and/or accumulation in the brain are very low.

In one aspect of the invention, farnesyl transferase inhibitors that cross the BBB are particularly useful for treating proteinopathies. In one embodiment, it is expected that farnesyl transferase inhibitors that are non-charged (e.g., not positively charged) and/or non-lipophilic may cross the BBB with higher efficiency than charged (e.g., positively charged) and/or lipophilic compounds. Therefore it will be appreciated by a person of ordinary skill in the art that some FTIs might readily cross the BBB. Alternatively, the FTI can be modified, for example, by the addition of various substitutents that would make them less hydrophilic and allow them to more readily cross the BBB.

Various strategies have been developed for introducing those drugs into the brain which otherwise would not cross the blood-brain barrier. Widely used strategies involve invasive procedures where the drug is delivered directly into the brain. One such procedure is the implantation of a catheter into the ventricular system to bypass the blood-brain barrier and deliver the drug directly to the brain. These procedures have been used in the treatment of brain diseases which have a predilection for the meninges, e.g., leukemic involvement of the brain (U.S. Pat. No. 4,902,505, incorporated herein in its entirety by reference).

Although invasive procedures for the direct delivery of drugs to the brain ventricles have experienced some success, they are limited in that they may only distribute the drug to superficial areas of the brain tissues, and not to the structures deep within the brain. Further, the invasive procedures are potentially harmful to the patient.

Other approaches to circumventing the blood-brain barrier utilize pharmacologic-based procedures involving drug latentiation or the conversion of hydrophilic drugs into lipid-soluble drugs. The majority of the latentiation approaches involve blocking the hydroxyl, carboxyl, and primary amine groups on the drug to make it more lipid-soluble and therefore more easily able to cross the blood-brain barrier.

Another approach to increasing the permeability of the BBB to drugs involves the intra-arterial infusion of hypertonic substances which transiently open the blood-brain barrier to allow passage of hydrophilic drugs. However, hypertonic substances are potentially toxic and may damage the blood-brain barrier.

Antibodies are another method for delivery of compositions of the invention. For example, an antibody that is reactive with a transferrin receptor present on a brain capillary endothelial cell, can be conjugated to a neuropharmaceutical agent to produce an antibody-neuropharmaceutical agent conjugate (U.S. Pat. No. 5,004,697, incorporated herein in its entirety by reference). The method is conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point of between about 8.0 to 11.0 (U.S. Pat. No. 5,527,527, incorporated herein in its entirety by reference).

A ligand-neuropharmaceutical agent fusion protein is another method useful for delivery of compositions to a host (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain barrier in a pharmaceutically active form.

The permeability of the blood brain barrier can be increased by administering a blood brain barrier agonist, for example bradykinin (U.S. Pat. No. 5,112,596, incorporated herein in its entirety by reference), or polypeptides called receptor mediated permeabilizers (RMP) (U.S. Pat. No. 5,268,164, incorporated herein in its entirety by reference). Exogenous molecules can be administered to the host's bloodstream parenterally by subcutaneous, intravenous, or intramuscular injection or by absorption through a bodily tissue, such as the digestive tract, the respiratory system, or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain barrier permeability is given later (e.g., between 30 minutes later and several hours later). This allows time for the drug to be absorbed in the gastrointestinal tract and taken up by the bloodstream before the agonist is given to increase the permeability of the blood-brain barrier to the drug. On the other hand, an agonist of blood-brain barrier permeability (e.g., bradykinin) can be administered before or at the same time as an intravenous injection of a drug. Thus, the term “co-administration” is used herein to mean that the agonist of blood-brain barrier and the exogenous molecule will be administered at times that will achieve significant concentrations in the blood for producing the simultaneous effects of increasing the permeability of the blood-brain barrier and allowing the maximum passage of the exogenous molecule from the blood to the cells of the central nervous system.

In other embodiments, an FTI can be formulated as a prodrug with a fatty acid carrier (and optionally with another neuroactive drug). The prodrug is stable in the environment of both the stomach and the bloodstream and may be delivered by ingestion. The prodrug passes readily through the blood brain barrier. The prodrug preferably has a brain penetration index of at least two times the brain penetration index of the drug alone. Once in the central nervous system, the prodrug, which preferably is inactive, is hydrolyzed into the fatty acid carrier and the farnesyl transferase inhibitor (and optionally another drug). The carrier preferably is a normal component of the central nervous system and is inactive and harmless. The compound and/or drug, once released from the fatty acid carrier, is active. Preferably, the fatty acid carrier is a partially-saturated straight chain molecule having between about 16 and 26 carbon atoms, and more preferably 20 and 24 carbon atoms. Examples of fatty acid carriers are provided in U.S. Pat. Nos. 4,939,174; 4,933,324; 5,994,932; 6,107,499; 6,258,836; and 6,407,137, the disclosures of which are incorporated herein by reference in their entirety.

The administration of the FTI may be for either prophylactic or therapeutic purposes. When provided prophylactically, the agent is provided in advance of disease symptoms. The prophylactic administration of the agent serves to prevent or reduce the rate of onset of symptoms of a proteinopathy. When provided therapeutically, the FTI is provided at (or shortly after) the onset of the appearance of symptoms of actual disease. In some embodiments, the therapeutic administration of the FTI serves to reduce the severity and duration of the disease.

The function and advantage of these and other embodiments of the present invention will be more fully understood from the examples described below. The following examples are intended to illustrate the benefits of the present invention, but do not exemplify the full scope of the invention.

### EXAMPLES

### Materials and Methods

Chemicals and reagents: DMEM and MEM were purchased from Gibco. All other reagents were purchased from Sigma. LNK-754 and Tipifarnib were synthesized for research purposes reported herein only.

Cell culture and immunocytochemistry: SH—SY5Y cells were grown in DMEM medium supplemented with 10% FBS and 1% pen/strep at 37° C. and 5% CO₂. Cells were differentiated with 10 μM retinoic acid for 48 hr, then treated with the either rapamycin (100 nM or 1 μM) or with 100 nM of either LNK-754-TS or Tipifarnib for 48-72 hr. Cells were then fixed with 4% paraformaldehyde and stained for LC3 (Novus biological, NB100-2331, dilution 1:800) followed by secondary Alexa-564 anti-Rabbit (A-11011).

Quantitative real-time PCR: Gene expression profiles were done by qPCR on series of known autophagy genes. RNA was extracted with Tri-reagent (Sigma), and cDNAs generated using iScript (Biorad). qPCR analysis was carried out in a 96 well plate using an iCycler (BioRad, Hercules, Calif.), and iQ SYBR Green Supermix (Biorad) according to the manufacturer's specifications.

Animals and treatments: Male and female human WT alpha-synuclein over-expressing transgenic mice³² at 6 months of age were given vehicle (10% beta-cyclodextrin) or LNK-754-TS (0.09, 0.9 and 9 mg/kg) per oral gavage twice daily for 3 months or animals at 7 months of age were given vehicle (2.5% beta-cyclodextrin) or LNK-754-TS (2 mg/kg) once every three days for 3 months. Male and female TAU transgenic mice expressing TAU441 bearing the missense mutations V337M⁵⁰ and R406W under the control of the murine Thy-1 promoter with a CB6×C57BL/6 background were 5 months old at the time when the oral treatment for three months with LNK-754-TS (0.9 and 0.09 mg/kg) as well as vehicle (2.5% beta-cyclodextrin) was started. Female human APP/PS1 (APP (London V7171)/PS1(A246E)) over-expressing transgenic mice were treated with LNK-754-TS (0.9 mg/kg) or vehicle (2.5% beta-cyclodextrin) for 2 months or 12 days.

Immunohistochemistry and quantification of stained cells: For evaluation of α-synuclein immunoreactivity (IR), 5 sagittal cryo-cut sections (10 μm slice thickness) from five different layers were used for counting of IR cells in the cortex and hippocampus. Brain sections were stained with a monoclonal human α-synuclein specific antibody (Alexis®; Cat#804-258-L001; dilution 1:5), followed by a secondary Ab Cy 2-Goat Anti-Rat (Jackson ImmunoResearch®; dilution 1:200). IR positive cells were quantified using specialized image analysis software (Image Pro Plus, version 4.5.1.29). For Tau transgenic animals, 5 μm thick coronal paraffin sections were stained with the monoclonal mouse anti-human TAU-antibodies (AT180-1:100; HT7-1:500) and visualized using an anti-mouse Cy3 secondary antibody (1:500, Jackson Laboratories®). Images were evaluated with ImageProPlus (version 6.2) image analysis software. For APP/PS1 transgenic animals sagittal hemisections (40 μm) were collected and processed for Aβ immunohistochemistry using an 6E10 antibody, Thioflavin-S staining. Primary antibodies were detected by the ABC method.

ELISA quantification of α-Synuclein in the α-Synuclein transgenic animals: Brain homogenate was centrifuged and the supernatant saved as fraction F1. The pellet was washed then resuspended and saved as fraction F2. Plates (Nunc, 464718) were coated with SYN-1 (1:1000, BD Transduction Labs, 610787). Monomeric recombinant α-synuclein was included as an internal standard. Biotinylated antibody FL-140 (1:300, Santa Cruz Biotechnology, sc-10717-B) and ExtrAvidin-Alkaline phosphatase (3:5000, Sigma, E2636) was added followed by pNPP substrate solution (Sigma, N1891). Raw absorbance (405 nm) was then normalized to the total protein concentration of each sample. In the APP/PS1 transgenic animal, brains were homogenized and the supernatant, Faction 1, was separated from the pellet. The pellets were further processed with addition of NP40 and Triton X-100. The supernatant was separated from the pellet as the insoluble membrane, Fraction 2, and was dissolved in 8M Guanidine. To quantify the amount of human Aβ-40 and Aβ-42, ELISA kits were used (The Genetics Company, Zurich, Switzerland).

Morris water maze (MWM) analysis of cognitive performance: In APP/PS1 transgenic animals, swimming behavior in a Morris Water Maze was videotaped and analyzed (Ethovision, Noldus, Wageningen, Netherlands). For mice, a place navigation test was used to locate the hidden platform in five blocks of three trials over three consecutive days. Each trial consists of a forced swim test of maximum 120 seconds, followed by 60 seconds of rest. The time each mouse needed for location of the platform was measured. For rats, a cued learning phase was first conducted, consisting of 3 trials per day for 5 days, using a visible platform of varying location. Each trial consisted of a forced swim test of maximum 60 seconds, followed by 10 minutes of rest. The time and path length each rat needed to locate the platform was measured.

Statistics: Data are represented as mean standard error of mean (SEM) with n>3 and significance at (p≦0.05). Normal distribution of measurement values were tested by paired T-test or one-way ANOVA, followed by a Newman-Keuis Multiple comparison posthoc test or Dunnett multiple comparison repeated measure posthoc test as indicated.

### Example 1

### Preparation of LNK-754-TS

The synthesis of LNK-754-TS (D-tartrate salt) is shown below in Schemes 1 and 2. The synthesis starts with the preparation of the ketone material 8. The synthesis of this material is shown in Scheme 1.

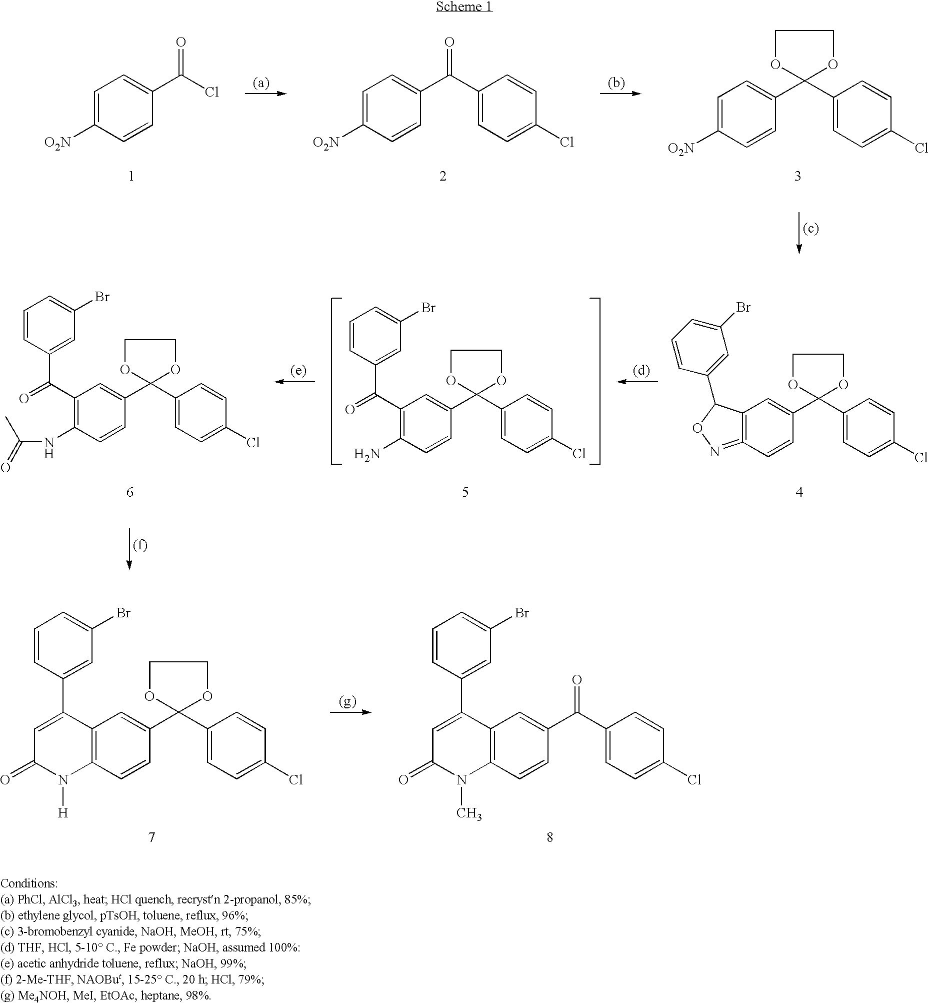

The GMP stage of the synthesis is shown in Scheme 2 and begins with a Sonogashira palladium-catalyzed coupling reaction [Step (h)]. In this reaction the trimethylsilyl acetylene group is coupled to the bromo-ketone (8).

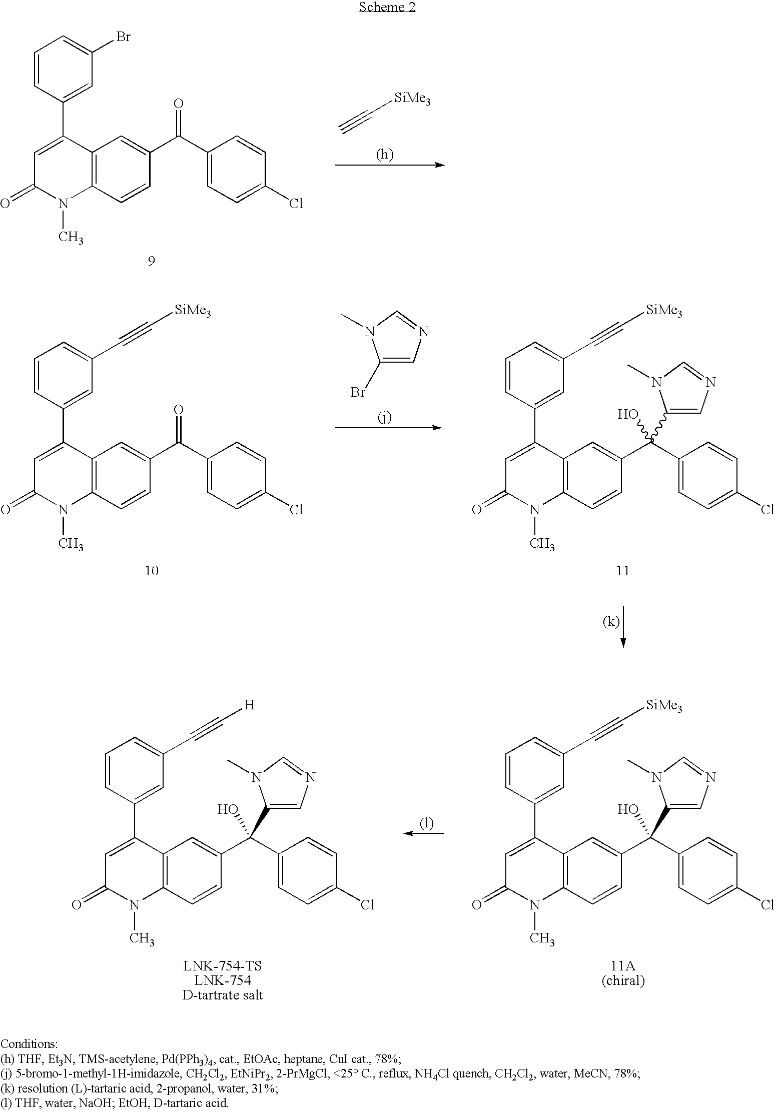

The resulting product (10) then undergoes a Grignard reaction [Scheme 2, Step (j)] with 5-bromo-1-methyl-1H-imidazole, giving 11 as a racemate. Purification of the racemate as its L-tartrate salt [Scheme 2, Step (k)] then gives chirally pure trimethylsilyl acetylene (11A). This compound is finally deprotected with sodium hydroxide and crystallized as its D-tartaric acid salt to produce LNK-754-TS [Scheme 2, Step (1)].

A narrative description of the manufacturing process, referring to Scheme 2, is provided below.

Step 1; Step (h): Tetrahydrofuran, 9, triethylamine, trimethylsilylacetylene, tetrakis (triphenylphosphino) palladium(II) chloride and copper(I) iodide were charged to a clean reaction vessel, under nitrogen, at 15-25° C. The reaction mixture was warmed to 47-52° C. with stirring and left at this temperature until the reaction was judged to be complete by HPLC (acceptance limit: not more than 1.0% (area) residual LNK5007 remaining).

The reaction mixture was cooled to 25-30° C. and treated with carbon and Celite, then stirred for several hours at 20-25° C. The mixture was filtered and washed with ethyl acetate. The filter cake of Celite and carbon was then suspended in ethyl acetate and stirred for 30-40 minutes at 30-40° C. The suspension was then filtered and washed with ethyl acetate.

The combined filtrates were then washed twice with sulphuric acid and diluted with water. The mixture was stirred in each case and allowed to settle, before draining the lower aqueous phase. The organic phase was successively washed with a solution of ammonium chloride in water, then with a solution of cysteine hydrochloride monohydrate and sodium hydrogen carbonate in water and finally with water alone. The organic phase was then evaporated in vacuo (0.7-0.9 bar) at below 50° C. to approximately 3 volumes and n-heptane is added, with stirring. The mixture was allowed to crystallize over 1 hour, then filtered, and washed with n-heptane. The filtered solid was dried to constant weight in vacuo, keeping the temperature below 40° C. A second crop may be obtained by evaporating the mother liquors.

Step 2; Step (j): Dichloromethane, 5-bromo-1-methyl-1H-imidazole and N-ethyldiisopropylamine were charged to a reaction vessel and the mixture was stirred at 15-25° C. to obtain a clear solution.

Isopropylmagnesium chloride in THF (20% w/w) was charged, keeping the temperature at 20-25° C., and the mixture stirred until the reaction was judged complete by GC (acceptance limit: 90-95% conversion or better). (In the event that reaction is not complete, further isopropyl magnesium chloride may be added to the reaction.)

A solution of 10 in dichloromethane was added over 5-10 minutes, keeping the temperature in the range 20-30° C. The flask that contained the 10 is rinsed with dichloromethane and the rinse transferred to the reaction vessel.

The reaction mixture was heated to reflux and left stirring until it was judged complete by HPLC (acceptance limit: not more than 10% 10 remaining).

The reaction mixture was cooled to 5-10° C. and washed with a solution of ammonium chloride in water. After separating the phases, the aqueous layer was back-washed with dichloromethane and the combined organic extract and dichloromethane wash were evaporated in vacuo. Acetonitrile was added in portions and the solvent evaporated, keeping the overall volume in the range 15-17 volumes. The residual mixture was stirred for 1 hour and cooled to 5-10° C., with stirring, to allow the product to crystallize.

The racemic 11 was filtered, washed with acetonitrile and dried to constant weight in vacuo at a temperature below 50° C.

The mother liquors were evaporated to approximately 3-3.5 volumes and allowed to crystallize, with stirring. The product was filtered, washed with acetonitrile and checked for purity by HPLC (acceptance limit: purity not less than 92.5% area). The second crop was then dried to constant weight in vacuo below 50° C.

Step 3; Step (k): Isopropanol and racemic 11 were heated to 75-80° C. until all of the solids dissolved.

A solution of L-tartaric acid in water, heated to 70-80° C., was added to the isopropanol solution, keeping the bulk reaction mixture at 75-80° C. After the addition was complete, the mixture was stirred at 78-80° C. for 30-40 minutes, then cooled over 30-60 minutes to 48-53° C.; where it was maintained for approximately 2 hours. Seed crystals of 11A (R-isomer) are added and the temperature ramped down in stages to 23-27° C.; at which point it was checked by chiral HPLC (acceptance limit: not less than 90% 11A). The crystalline product was filtered and washed with isopropanol and air-dried. The wet cake was suspended in isopropanol and heated to 50-55° C. for 1-1.5 hours; then cooled to 20-25° C. and stirred for 3-4 hours.

The crystalline product was filtered and rinsed with isopropanol and air-dried before analysis by HPLC (acceptance limit: not less than 96% 11A (R-isomer); not less than 97% area chemical purity).

The product was dried to constant weight in vacuo at below 60° C.

A second crop may be obtained from the mother liquors with the same acceptance criteria as for the first crop.

Step 4; Step (I): Tetrahydrofuran, deionized water and 11A were charged to a reaction vessel and stirred at 20-25° C. A solution of sodium hydroxide in deionized water was added and the mixture was stirred at 20-30° C. until the reaction was judged complete by HPLC (acceptance limit: not more than 0.5% area of 11A remaining in the reaction mixture.)

The organic layer was separated and the aqueous layer extracted twice more with 2-methyltetrahydrofuran. The combined organic extracts were washed with a solution of cysteine hydrochloride and sodium hydrogen carbonate in water. After confirming that the pH was not less than 7, the organic layer was separated and washed with a solution of sodium chloride in deionized water. The organic layer was again separated and treated with a mixture of Celite and activated carbon then stirred for 1-1.5 hours at ambient temperature. The resulting suspension was filtered and washed with 2-methyltetrahydrofuran and the filtrate was evaporated to dryness in vacuo below 60° C. To the residue was added isopropanol and evaporation to dryness was repeated before analysis by HPLC (acceptance limit: not less than 96% LNK-754.)

LNK-754 free-base and absolute ethanol (13 weight) were charged to a reactor and heated to 50° C. In order to dissolve the solid, it was necessary to add deionized water until a solution formed. The solution was hot filtered to a second (clean) vessel and heated to reflux.

In a separate vessel, D-tartaric acid and water were heated to 50-60° C. until a solution forms. This solution was hot-filtered and transferred to the vessel containing LNK-754 free-base solution at reflux. The solution was allowed to cool to 5-10° C. at which point an amorphous solid began to precipitate. The mixture was warmed to 15-20° C. with stirring and held at this temperature to allow the mixture to crystallize. The solid was filtered and washed with ethanol. The wet cake was suspended in ethyl acetate and the solvent was partially removed by distillation under partial vacuum at 30-40° C. Aliquots of ethyl acetate were then charged and distilled from the mixture under partial vacuum at 30-40° C. (azeotropic removal of water).

The mixture was cooled to 20-25° C. and stirred for one hour, then filtered and washed twice with ethyl acetate, before drying in vacuo at 40-45° C.

The dried solid LNK-754-TS was suspended in ethyl acetate which was removed by distillation at atmospheric pressure. The suspension was cooled to 20-25° C. and held for one hour, then filtered, washed with ethyl acetate again and dried to constant weight in vacuo at 40-45° C. to result in the final drug substance. The XRPD fingerprint and peak data are consistent with polymorph Form A (U.S. Pat. No. 6,734,308). Table 1A below shows a listing of the more prominent 2θ angles, d-spacings and relative intensities.

TABLE 1A
X-ray Powder Diffraction, 2θ Angles, D-spacing and Relative
Intensities (Using Cu Kα Radiation) for LNK-754-TS.

| Pos. | Height | FWHM | d-spacing | Rel. Int. |
|---|---|---|---|---|
| [°2θ.] | [cts] | [°2θ.] | [Å] | [%] |
| 3.6301 | 127364.50 | 0.1020 | 24.32031 | 100.00 |
| 6.2576 | 3760.20 | 0.1065 | 14.11303 | 2.95 |
| 7.2584 | 1304.58 | 0.0900 | 12.16926 | 1.02 |
| 9.5874 | 781.74 | 0.0900 | 9.21764 | 0.61 |
| 10.8584 | 602.49 | 0.1020 | 8.14133 | 0.47 |
| 12.2790 | 184.01 | 0.1020 | 7.20244 | 0.14 |
| 12.5290 | 569.68 | 0.0900 | 7.05929 | 0.45 |
| 13.7530 | 398.75 | 0.0900 | 6.43365 | 0.31 |
| 14.5010 | 601.37 | 0.0900 | 6.10343 | 0.47 |
| 15.9857 | 2320.21 | 0.0999 | 5.53976 | 1.82 |
| 17.2062 | 2846.28 | 0.1020 | 5.14945 | 2.23 |
| 17.5658 | 4329.29 | 0.1814 | 5.04481 | 3.40 |
| 18.8667 | 767.40 | 0.1020 | 4.69981 | 0.60 |
| 19.1967 | 2954.02 | 0.1900 | 4.61975 | 2.32 |
| 19.7291 | 440.77 | 0.0816 | 4.49626 | 0.35 |
| 20.3734 | 5437.80 | 0.0849 | 4.35551 | 4.27 |
| 22.1461 | 1725.99 | 0.0816 | 4.01072 | 1.36 |
| 23.4770 | 935.34 | 0.0900 | 3.78627 | 0.73 |
| 23.8994 | 309.07 | 0.0612 | 3.72030 | 0.24 |
| 25.0879 | 2305.07 | 0.0263 | 3.54669 | 1.81 |
| 25.5106 | 561.97 | 0.1020 | 3.48887 | 0.44 |
| 26.6730 | 1024.09 | 0.0900 | 3.33940 | 0.80 |
| 27.9740 | 947.05 | 0.0900 | 3.23230 | 0.74 |
| 28.1860 | 870.19 | 0.0900 | 3.16349 | 0.68 |
| 28.4920 | 1074.91 | 0.0900 | 3.13021 | 0.84 |
| 28.9170 | 833.18 | 0.0900 | 3.08516 | 0.65 |
| 29.9370 | 852.00 | 0.0900 | 2.98233 | 0.67 |

### Example 2

### Preparation of Zarnestra®

Zarnestra® can be prepared according to the procedure described in WO 97/21701.

### Example A.1

1a) N-Phenyl-3-(3-chlorophenyl)-2-propenamide (58.6 g) and polyphosphoric acid (580 g) were stirred at 100° C. overnight. The product was used without further purification, yielding quant. (±)-4-(3-chlorophenyl)-3,4-dihydro-2(1H)-quinolinone (interm. 1-a).

1b) Intermediate (1-a) (58.6 g), 4-chlorobenzoic acid (71.2 g) and polyphosphoric acid (580 g) were stirred at 140° C. for 48 hours. The mixture was poured into ice water and filtered off. The precipitate was washed with water, then with a diluted NH₄OH solution and taken up in DCM. The organic layer was dried (MgSO₄), filtered off and evaporated. The residue was purified by column chromatography over silica gel (eluent:CH₂Cl₂/CH₃OH/NH₄OH 99/1/0.1). The pure fractions were collected and evaporated, and recrystallized from CH₂Cl₂/CH₃OH/DIPE, yielding 2.2 g of (±)-6-(4-chlorobenzoyl)-4-(3-chlorophenyl)-3,4-dihydro-2(1H)-quinolinone (interm. 1-b, mp. 194.8° C.).

1c) Bromine (3.4 ml) in bromobenzene (80 ml) was added dropwise at room temperature to a solution of intermediate (1-b) (26 g) in bromobenzene (250 ml) and the mixture was stirred at 160° C. overnight. The mixture was cooled to room temperature and basified with NH₄OH. The mixture was evaporated, the residue was taken up in ACN and filtered off. The precipitate was washed with water and air dried, yielding 24 g (92.7%) of product. A sample was recrystallized from CH₂Cl₂/CH₃OH/DIPE, yielding 2.8 g of 6-(4-chlorobenzoyl)-4-(3-chlorophenyl)-2(1H)-quinolinone; mp. 234.8° C. (interm. 1-c).

1d) Iodomethane (6.2 ml) was added to a mixture of intermediate (1-c) (20 g) and benzyltriethylammonium chloride (5.7 g) in tetrahydrofuran (200 ml) and sodium hydroxide (ION) (200 ml) and the mixture was stirred at room temperature overnight. ethyl acetate was added and the mixture was decanted. The organic layer was washed with water, dried (MgSO₄), filtered off and evaporated till dryness. The residue was purified by column chromatography over silica gel (eluent CH₂Cl₂/CH₃OH/NH₄OH 99.75/0.25/0.1). The pure fractions were collected and evaporated, yielding 12.3 g (75%) of 6-(4-chlorobenzoyl)-4-(3-chlorophenyl)-1-methyl-2(1H)-quinolinone; mp. 154.7° C. (interm. 1-d).

In a similar way, but starting from intermediate (1-b), (±)-6-(4-chlorobenzoyl)-4-(3-chlorophenyl)-3,4-dihydro-1-methyl-2(1H)-quinolinone (interm 1-e) was prepared.

### Example A.3

3a) Butyllithium (30.1 ml) was added slowly at −78° C. to a solution of N,N-dimethyl-1H-imidazol-1-sulfonamide (8,4 g) in tetrahydrofuran (150 ml) and the mixture was stirred at −78 C for 15 minutes. Chlorotriethylsilane (8.1 ml) was added and the mixture was stirred till the temperature reached 20° C. The mixture was cooled till −78° C., butyllithium (30.1 ml) was added, the mixture was stirred at −78° C. for 1 hour and allowed to reach −15° C. The mixture was cooled again till −78° C., a solution of 6-(4-chlorobenzoyl)-1-methyl-4-(3-chlorophenyl)-2(1H)-quinolinone (15 g) in tetrahydrofuran (30 ml) was added and the mixture was stirred till the temperature reached 20° C. The mixture was hydrolyzed and extracted with ethyl acetate. The organic layer was dried (MgSO₄), filtered off and evaporated till dryness. The product was used without further purification, yielding (±)-4-[(4-chlorophenyl)(1,2-dihydro-1-methyl-2-oxo-4-(3-chlorophenyl)-6-quinolinyl)hydroxymethyl]-N,N-dimethyl-2-(triethylsilyl)-1H-imidazole-1-sulfonamide (interm. 3-a).

A mixture of intermediate (3-a) (26 g) in sulfuric acid (2.5 ml) and water (250 ml) was stirred and heated at 110° C. for 2 hours. The mixture was poured into ice, basified with NH₄OH and extracted with DCM. The organic layer was dried (MgSO₄), filtered off and evaporated till dryness. The residue was purified by column chromatography over silica gel (eluent: CH₂Cl₂/CH₃OH/NH₄OH 99/1/0.2). The pure fractions were collected and evaporated, yielding 2.4 g (11%) of (±)-4[(4-chlorophenyl)(1,2-dihydro-1-methyl-2-oxo-4-(3-chlorophenyl)-6-quinolinyphydroxymethyl-N,N-dimethyl-1H-imidazole-1-sulfonamide (interm. 3-b).

### Example A.4

Compound (3) (3 g) was added at room temperature to thionyl chloride (25 ml). The mixture was stirred and refluxed at 40° C. overnight. The solvent was evaporated till dryness. The product was used without further purification, yielding (±)-4-(3-chlorophenyl)-1-methyl-6-[(4-chlorophenyl)-1-(4-methyl-4H-pyrrol-3-yl)ethyl]-2(1H)-quinolinone hydrochloride (interm. 4).

### Example B.13

NH₃ (aq.) (40 ml) was added at room temperature to a mixture of intermediate 4 (7 g) in THE (40 ml). The mixture was stirred at 80° C. for 1 hour, then hydrolyzed and extracted with DCM. The organic layer was separated, dried (MgSO₄), filtered and the solvent was evaporated. The residue was purified by column chromatography over silica gel (eluent:toluene/2-propanol/NH₄OH 80/20/1). The pure fractions were collected and the solvent was evaporated, yielding (±)₆-[amino(4-chlorophenyl)(1-methyl-1H-imidazol-5-yl)methyl]-4-(3-chlorophenyl)-1-methyl-2(1H)-quinolinone. This racemic compound can be separated into it single enantiomers using techniques known in the art.

### Example 3

### Dosing of LNK-754-TS In Vivo

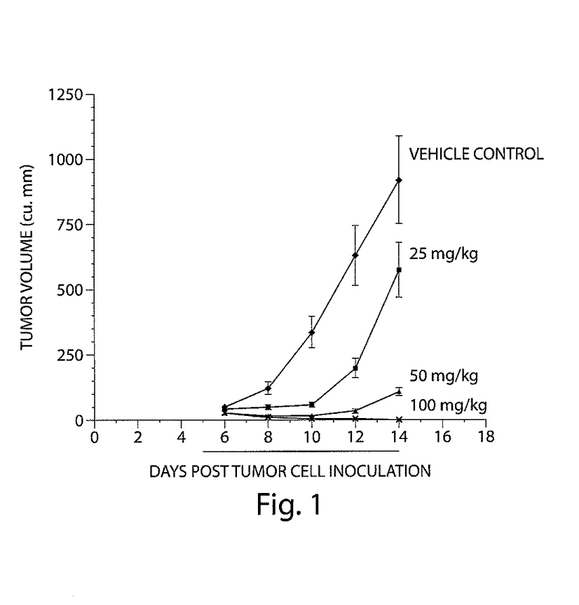

Farnesyl transferase inhibitors were originally developed to target the oncogenic protein Ras and have been dosed at high doses to achieve an almost total inhibition of Ras farnesylation. Ras as a target and the high dosing and high degree of the inhibition of Ras farnesylation are based on targeting cancer cells for cell death. The doses of FTIs used are thus significantly higher in cancer therapeutics than the doses that are efficacious in neurodegeneration applications. Evidence for this in mice is given in FIGS. 1-3. In FIG. 1 is shown the efficacy of LNK-754-TS in a xenograft tumor mouse model. The lowest dose tested, 25 mg/kg, shows borderline efficacy against tumor growth in this model and is significantly higher than efficacious doses in PD and AD transgenic mouse models. Doses below 25 mg/kg were not tested in the xenograft model, due to lack of efficacy.

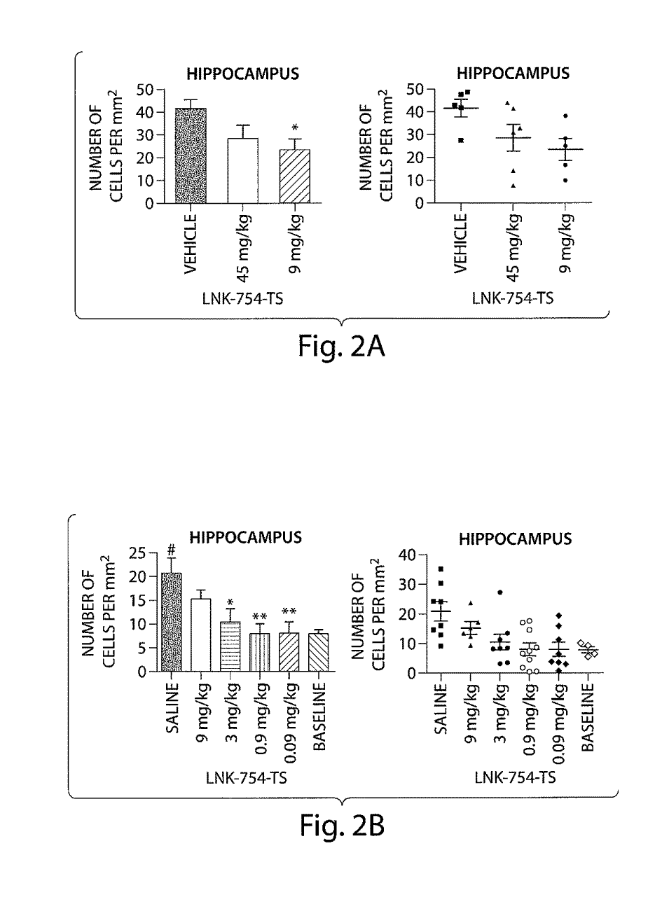

In FIG. 2 is shown efficacy data for LNK-754-TS in the Masliah D-line transgenic α-synuclein mouse (an accepted model of synucleinopathies). Two trials are shown, the first (FIG. 2A) at higher doses of LNK-754-TS: 45 mg/kg and 9 mg/kg. In this trial, the highest dose of LNK-754-TS, 45 mg/kg, is not significantly effective in lowering the number of α-synuclein positive neurons in the brains of treated mice, while the lower dose, 9 mg/kg, shows a significant reduction in the number of α-synuclein positive neurons. The second trial (FIG. 2B) explores the low dose range for efficacy in the α-synuclein models. Here, doses start as low as 0.9 mg/kg, and extend through 9 mg/kg, all below the efficacious dose range in the mouse oncology model.

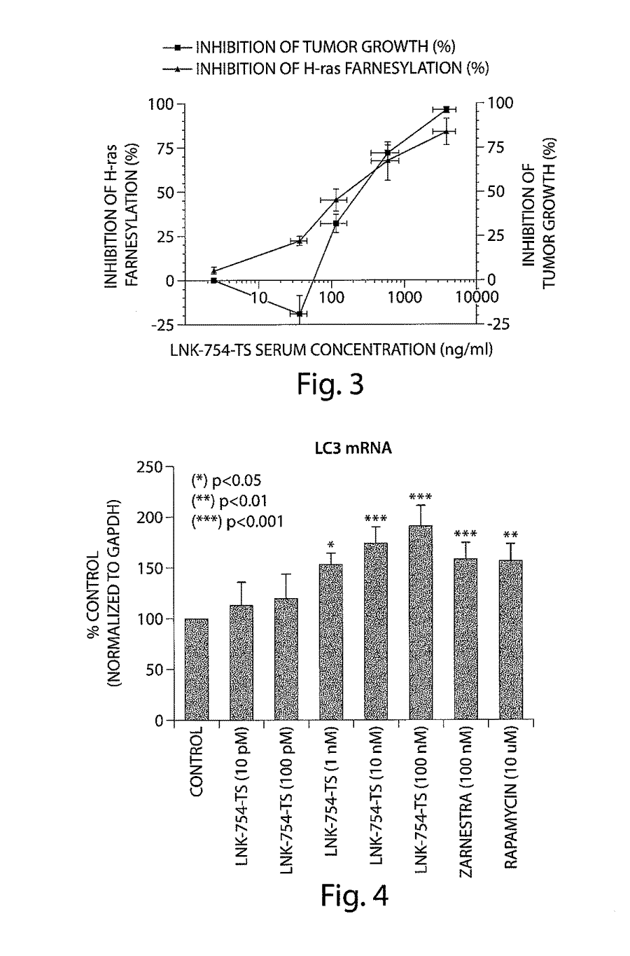

Further data supporting the stark difference in dosing levels for efficacy in oncology and synucleinopathies is shown in FIG. 3 and Table 2A below. In the experiment shown in FIG. 3, a xenograft model is once again used, but there is continuous infusion of LNK-754-TS, and thus a steady state concentration of drug in the plasma and tissues. In this experiment, it is necessary to achieve both continuous serum levels above 100 ng/ml (AUC), and a resultant minimum of 50% inhibition of Ras farnesylation in tumor tissue, in order to observe significant inhibition of tumor growth

TABLE 2A
Pharmacokinetic parameters in mice for LNK-754-TS.

| Dose | | | # | AUC | | |
|---|---|---|---|---|---|---|
| mg/kg | Vehicle | regimen | subj | ng/ml | Tmax | Cmax ng/ml |
| 9 | 20% beta- | BID day 1 | 3 | 2099 | 1 | 1385 |
| | cyclodextrin | | | | | |
| 9 | 20% beta- | BID day 5 | 3 | 2628 | 1 | 1485 |
| | cyclodextrin | | | | | |
| 0.09 | 5% beta- | QD day 1 | 3 | 0.63 | 0.5 | 0.61 |
| | cyclodextrin | | | | | |
| 0.9 | 5% beta- | QD day 1 | 3 | 34.57 | 0.5 | 31.07 |
| | cyclodextrin | | | | | |

In the experimental data represented in Table 2A, a different method of drug delivery is used (oral) than in the experiment represented in FIG. 3. The best way to compare the relative coverage of the two delivery methods (oral and continuous infusion) is by comparing area-under-the-curve (AUC) values. PK analysis of oral dosing of LNK-754-TS in mice is shown in the table. We can compare the calculated AUC values for the continuous infusion oncology study presented in FIG. 3 and the AUC values associated with the synuclein model doses in the table. With a minimal continuous serum level of 100 ng/ml, there should be a resultant minimal efficacious AUC of approximately 2400 ng/ml. As shown in the table, the AUC of orally dosed LNK-754-TS at 9 mg/kg BID is between 2000 and 2600 ng/ml. The AUCs of orally dosed LNK-754-TS at 0.9 mg/kg and at 0.09 mg/kg QD are 34.6 and 0.63 ng/ml, respectively. The 9 mg/kg BID dose, which is at the high end of doses showing efficacy in the α-synuclein model, is roughly equivalent in AUC to the lowest efficacious dose in the xenograft cancer model. The 0.9 and 0.09 mg/kg doses, which are efficacious in the α-synuclein model dosed both BID and QD, have QD dosed AUCs that are significantly below the efficacious range in the xenograft model (i.e., they should be below 10 ng/ml on the x-axis in FIG. 3—with 10 ng/ml calculating at 240 ng/ml AUC). The BID dosing should only increase the AUC by several fold at most, thus resulting in values for these two doses far below the levels of LNK-754-TS needed to achieve 50% inhibition of Ras farnesylation.

In conclusion, the mouse data supports that efficacious dosing of LNK-754-TS in the α-synuclein model in mice (and also in the AD models tested) starts well below the lowest oncology efficacious dose, and that efficacy is reduced as dosing enters the efficacious range in the oncology model.

### Example 4

### Dosing of LNK-754-TS In Vitro

### Autophagy

Currently, the dose-response experiments with LNK-754-TS are in the SH—SY5Y cell line and show that at doses of LNK-754-TS between 1 and 100 nM, there are significant increases in the levels of mRNA of LC3, a key autophagy-associated protein (FIG. 4). Such increases in LC3 mRNA levels are associated in the literature with stimulation of macroautophagy. This supports the hypothesis that at doses as low as 1 nM in this in vitro system there is stimulation of autophagy in these cells. Zamestra® also works in this assay (at 100 nM concentration). Rapamycin, tested at a concentration where it is reported to stimulate autophagy, is a positive control (FIG. 4).

Ras vs. HDJ2 Farnesylation

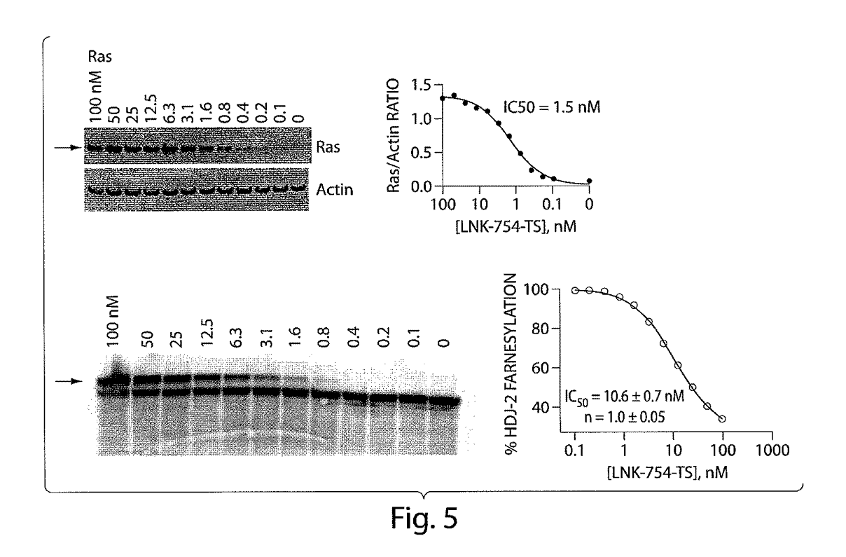

Using the same cell line treated with LNK-754-TS in FIG. 4, different IC₅₀ values are observed for the inhibition of farnesylation of two different protein FTAse substrates, Ras and HDJ2 (FIG. 5). It is important to emphasize that there is not a good match between concentrations of FTIs required for inhibition of the farnesylation of specific substrates in vitro and in viva (for a variety of reasons). In this particular set of experiments, with continuous exposure of drug to the cell line over long periods, while Ras farnesylation is inhibited at an average IC₅₀ of 1 nM, HDJ2 farnesylation is inhibited at an IC₅₀ of 10 nM. This supports the hypothesis that different concentrations of FTIs will target different sets of farnesylated substrate proteins, with different biological results in different concentration ranges of drug treatment. The non-Ras substrate proteins could include non-CaaX—CO₂H proteins such as UCH-L1, or alternate CaaX-CO₂H substrate proteins.

### Example 5

### Effect of LNK-754-TS on Non-farnesylated Ras Levels in LNK-754-TS Treated Mice

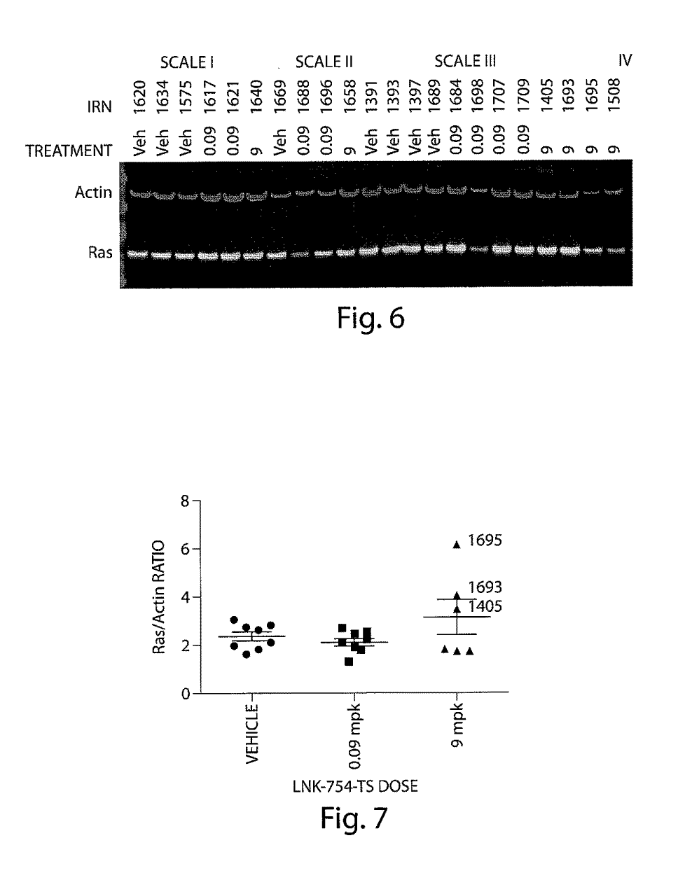

The level of inhibition of Ras in brain by LNK-754-TS, dosed at an efficacious dose for efficacy in animal models of proteinopathy-dependent neurodegeneration, was investigated. Alpha-synuclein transgenic mice were treated for 3 months b.i.d. with vehicle or LNK-754-TS at 0.09 mg/kg or 9 mg/kg. Cortical tissue was extracted and homogenized, followed by isolation of soluble/cytosolic proteins in detergent-free buffer (50 mM Tris-HCl pH 7.4, 140 mM NaCl, 2 mM EDTA, Protease inhibitor cocktail) by centrifugation. 15 micrograms of protein lysate was analyzed per lane of SDS-PAGE gel, and immunoblotted for Ras and actin (FIG. 6). Densitometry was used to quantify the Ras/actin ratio for each sample, and results were plotted (FIG. 7). No significant differences in soluble Ras/actin level were detected between groups, using one-way ANOVA or student's T-test. Thus, doses of LNK-754-TS able to improve the pathology in both PD and AD transgenic models had no significant effect on Ras farnesylation in the target tissue of brain. This contrasts with what is observed in xenograft cancer models, where inhibition of Ras farnesylation by high dose FTIs is directly correlated with efficacy (FIG. 3 and Example 3).

### Example 6

### Evaluating the Efficacy of Inventive Compounds on Reducing Phospho-Tau Accumulation in TAU Transgenic Mice

Like α-synuclein, tau is a highly expressed cytosolic protein and is an autophagy substrate (Hamano et al., Eur. J. Neurosci. 27(5):1119-30, Mar. 2008). Cytosolic tau aggregates are characteristic of Alzheimer's disease (AD) (neurofibrillary tangles) and of frontotemporal dementia (FTD). Appearance of tau aggregates (detected by the presence of specific phosphorylated tau forms that correlate with disease) is correlated with brain pathology in both humans and animal models (and is also induced by autophagy inhibition via a reduction of p62 expression; Ramesh et al., J. Neurochem. 106(1):107-20, Jul. 2008). Autophagy stimulation by LNK-754-TS could thus be expected to reduce levels of pathological, phosphorylated tau in appropriate animal models. We chose to study 0.5 month-old TAU transgenic (tg) mice with a CB6×C57BL/6 background which express TAU441 bearing the missense mutations V337M and R406W under the regulatory control of the murine Thy-1 promoter, where amygdala is the primary site of tau deposition and, therefore the primary behavioral abnormality is depression.

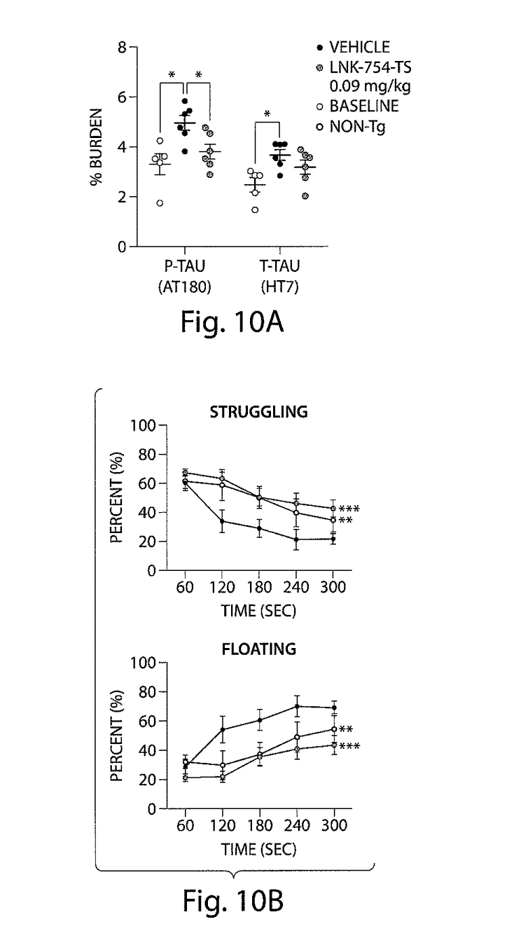

This study was designed to evaluate the effects of a treatment with LNK-754-TS dosed at 0.09 mg per kg on behavior, TAU and TAU-pT231 levels, and brain morphology of TAU441 Tg mice. Histological evaluations were performed to quantitatively evaluate TAU pathology. TAU depositions were determined using the monoclonal TAU-antibodies AT180 and HT7. AT180 recognizes phosphorylated TAU and tangle-like formations (the epitope of this antibody is the phosphorylated Thr231 residue), HT7 normal human TAU and phosphorylated TAU (the epitope of this antibody has been mapped to a region between residues 159 and 163 of human TAU). 5 μm thick coronal paraffin sections from each of the five different layers were stained with the above-described monoclonal mouse anti-human TAU-antibodies (AT180 at 1:100; HT7 at 1:500) and visualized using an anti-mouse Cy3 secondary antibody (1:500, Jackson Laboratories). Tiled images were recorded using a PCO Pixel Fly camera mounted on a Nikon E800 with a StagePro software controlled table and an exposure time of 300 msec for AT180 and HT7 fluorescence at 200-fold magnification. Afterwards images were evaluated with ImageProPlus (version 6.2) image analysis software (FIG. 10A).

### Results

Measured region areas of the amygdala were highly constant throughout all investigated brains which exclude negative effects on tissue in immunohistochemical procedural steps (e.g., irregular shrinkage, different cutting circumstances). Both HT7 and AT180 IR increased age-dependently in the amygdala between baseline at five months of age and 8 months at sacrifice: specifically, in the amygdala, phosphorylated Tau was significantly decreased after LNK-754-TS treatment (t-test: p=0.02 versus vehicle; FIG. 10A). HT7 immunoreactive total TAU levels were not significantly reduced on treatment. Qualitatively the reduction of AT180 immunoreactive phosphorylated Tau in the amygdala was visible as a reduction in the number of immunoreactive cells. The pattern of perinuclear staining in immunoreactive cells was not apparently different from those seen in cells of vehicle controls. The number of affected cells was comparable to those of baseline animals (FIG. 10A).

### Example 7

### Evaluating the Efficacy of Inventive Compounds on Reversing TAU-Dependent Depression in TAU Transgenic Mice

Tests relevant to depression-like behaviors in rodents are primarily stress-induced reductions in avoidance or escape, termed behavioral despair. One of the most widely used animal tests for depression is the Porsolt forced swim task (Porsolt et al., Arch. Int. Pharmacodyn. Ther. 229(2):327-36, 1977; Porsolt et al., Eur. J. Pharmacol. 47(4):379-91, 1978). This study was designed to evaluate the effects of treatment with LNK-754 on behavior of TAU441 transgenic mice. At start of the treatment, the animals were 5 months old. Untreated non-transgenic animals of the same age were tested and sacrificed serving as the baseline group. Mice received vehicle or LNK-754-TS at a dose of 0.09 mg per kg, 7 days a week for 90 days. In the last week of the treatment period and before sacrifice, mice were evaluated using the Porsolt forced swim task (FIG. 10B).

### Results

After 120 seconds of testing until the end of the trial period, animals treated with LNK-754-TS showed significantly less floating (p<0.001), paired with a higher percentage of struggling behavior compared to vehicle treated animals, which suggests therapeutic correction of the ptau-dependent depressive phenotype by LNK-754-TS (FIG. 10B). Remarkably, animals treated with LNK-754-TS behaved similar to non-transgenic mice (FIG. 10B).

### Example 8

### Stimulation of Cellular Autophagy with an FTI

Farnesyltransferase (FTase) inhibition reduces accumulation of α-synuclein in cell culture (Liu, Z., et al. Proc Natl Acad Sci USA 106, 4635-4640 (2009). Furthermore, LNK-754-TS reduces levels of alpha-synuclein in transgenic mouse models of PD. The possibility that autophagy stimulation was responsible was investigated based on two facts: (1) neuronal α-synuclein is degraded in part by autophagy (Vogiatzi, T., et al. J. Biol Chem (2008)) and (2) α-synuclein clearance is stimulated by rapamycin, which is known to stimulate autophagy by inhibiting mTOR (Webb, J. L., et al. J Biol Chem 278, 25009-25013 (2003)).

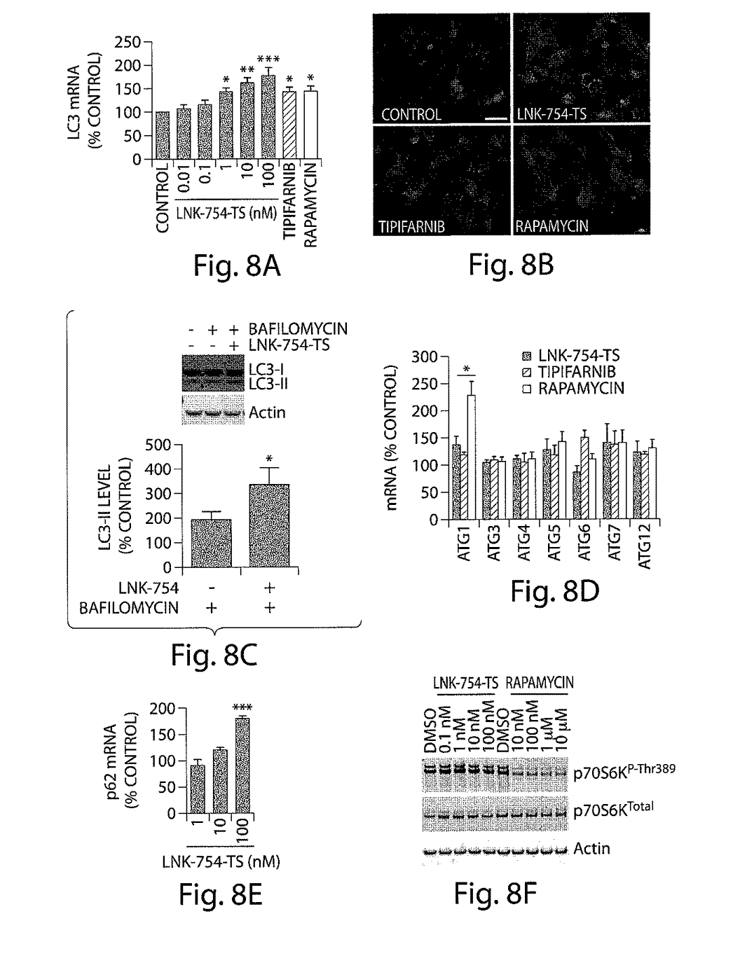

Autophagy was measured in a neuroblastoma cell culture system by three distinct approaches: quantitation of autophagy-related mRNA's, immunofluorescence microscopy of autophagosomes, and biochemical detection of the microtubule-associated protein 1 light chain 3 (LC3) a key protein that is required for autophagosome formation. Differentiated human neuroblastoma cells (SH—SY5Y) were treated for 72 hr with LNK-754-TS (0.01-100 nM), Zarnestra® (also referred to herein as tipifarnib) (100 nM) or rapamycin (1 μM). LC3 transcript, which encodes a key, membrane associated protein component of the autophagosome (Kirisako, T., et al. J Cell Biol 147, 435-446 (1999)) was upregulated by all three compounds (FIG. 8a); most potently by LNK-754-TS. All three compounds also caused a distinct increase in the number of LC3-positive puncta (FIG. 8b), consistent with an increased number of autophagosomes (Klionsky, D. J., et al. Autophagy 4, 151-175 (2008) and increased autophagy.

The observed increase in LC3-positive autophagosomes could result, in principle, from either an increased flux through the autophagy pathway or decreased autophagosome degradation (Pankiv, S., et al. J Biol Chem 282, 24131-24145 (2007); Kamada, Y., et al. J Cell Biol 150, 1507-1513 (2000)). The latter possibility is inconsistent with the observation that treatment with LNK-754-TS alone did not cause accumulation of either the cytosolic form of LC3 protein, LC3-I, or the autophagosome-associated, lipid-conjugated form, LC3-II, itself an autophagy substrate. In order to ascertain an increase in autophagic flux, cells were co-treated with LNK-754-TS and an inhibitor of autophagosome-lysosome fusion, bafilomycin A1 (10 nM). Bafilomycin treatment alone caused a 100% increase in the amount of LC3-II, consistent with the fact that it inhibits autophagosome degradation (FIG. 8c). The combination of bafilomycin and LNK-754-TS caused an additional 75% increase in LC3-II over bafilomycin alone (FIG. 8c) suggesting that LNK-754-TS increases autophagic flux, in part by acting upstream of autophagosome-lysosome fusion (Pan, J., et al. Cancer Biol Ther 7, 1679-1684, 2008; Kamada, Y., et al. J Cell Biol 150, 1507-1513,2000). Taken together, the data indicated that LNK-754-TS stimulates both parts of the autophagy pathway: autophagosome synthesis and autophagosome degradation.

Finally, LNK-754-TS (100 nM) treatment of SH—SY5Y cells induced upregulation of the transcript encoding p62 (FIG. 8e), which interacts with LC3-II and polyubiquitin chains and is required for autophagy (Pankiv, S., et al. J Biol Chem 282, 24131-24145 (2007)).

The mechanism of autophagy stimulation by LNK-754-TS appears distinct from that of the drug rapamycin. Rapamycin is a well-characterized autophagy stimulator that acts through inhibition of mTOR, a kinase involved in nutrient signaling and regulation of cell growth and survival. Like LNK-754-TS, rapamycin (100 nM) treatment of SH—SY5Y cells increased LC3-II protein levels in the presence of bafilomycin A1 (FIG. 8c). To further contrast the mechanism of autophagy stimulation by LNK-754-TS to that of rapamycin, a collection of mRNA transcripts of autophagy proteins were measured (FIG. 8d). Selected mRNAs from untreated SH—SY5Y cells were compared to mRNAs from cells treated with LNK-754-TS (100 nM), tipifarnib (100 nM), or rapamycin (1 μM). Rapamycin, but not tipifarnib or LNK-754-TS, caused an increase in the transcript encoding Atg1, an autophagy protein that forms a key link with the mTOR pathway (Kamada, Y., et al. J Cell Biol 150, 1507-1513 (2000)) (FIG. 8d). Furthermore, unlike rapamycin, LNK-754-TS did not inhibit phosphorylation of p70 S6 kinase (S6K), a downstream target of the mTOR pathway (FIG. 8J). Together, these findings suggest that LNK-754-TS stimulates autophagy by an mTOR-independent pathway distinct from that of rapamycin.

### Example 9

### Low Dose FTI Treatment Shows Efficacy in Transgenic Models of Neurodegeneration

LNK-754-TS reduces α-synuclein accumulation in human WT-α-synuclein transgenic mice.

The effect of LNK-754-TS on α-synuclein accumulation was investigated in a well-characterized transgenic mouse model of progressive aggregation and accumulation of human α-synuclein in the cortex and hippocampus (Masliah, E., et al. Science 287, 1265-1269 (2000)). Stimulation of autophagy in this mouse, by local expression of virally-encoded beclin (Pickford, F., et al. J Clin Invest 118, 2190-2199 (2008)), has been reported to reduce α-synuclein accumulation.

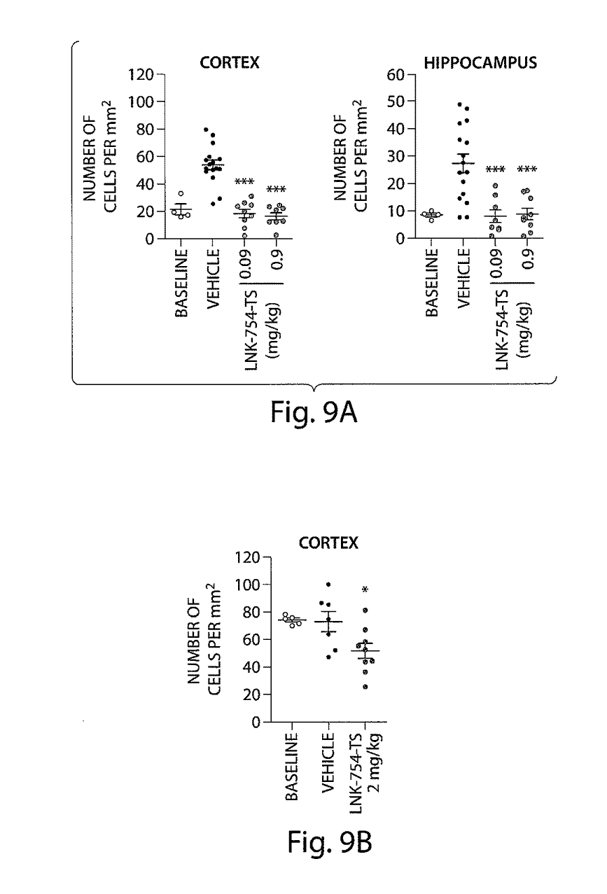

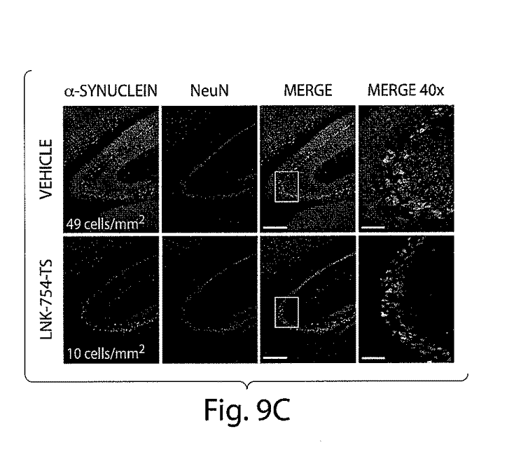

After dosing with LNK-754-TS for three months (twice daily at 0.09 mg/kg or 0.9 mg/kg), α-synuclein accumulation in the brain was analyzed by immunohistochemical (human specific α-synuclein immunoreactivity) and biochemical (α-synuclein ELISA) means. Both of these measures, which were correlated on a per animal basis, showed that LNK-754-TS treatment clearly reduced α-synuclein accumulation (FIG. 9a and FIG. 9c). In fact, the level of α-synuclein post-treatment was comparable to, or below that measured at the beginning of treatment (FIG. 9a). None of the treated animals showed any evidence of drug-dependent toxicity. There was no evidence of neuronal loss (NeuN staining and brain volume were unchanged), synaptic damage (synaptophysin staining was unchanged), or astrocytosis (GFAP staining was unchanged).

In order to test whether autophagy stimulation is responsible for α-synuclein clearance by LNK-754-TS, a second trial was designed to answer two clinically meaningful questions: (1) can LNK-754-TS treatment reduce preexisting α-synuclein deposits? and (2) is intermittent treatment effective? Treatment with LNK-754-TS was initiated at a time when α-synuclein immunoreactivity in the cortex had plateaued (FIG. 9b). After three months of intermittent dosing with LNK-754-TS (one dose (2 mg/kg), every 72 hours), α-synuclein immunoreactivity was significantly lower than at the outset of treatment (FIG. 9b), suggesting that pre-existing α-synuclein aggregates had been cleared. This finding is consistent with the proposed mechanism of autophagy stimulation and has important implications for clinical trials.

### LNK-754-TS Reduces Phosphorylated-TAU Accumulation in TAU Transgenic Mice.

Like α-synuclein, tau is a highly expressed protein that aggregates in the neuronal cytosol and can be cleared by autophagy (Hamano, T., et al. Eur J Neurosci 27, 1119-1130 (2008)). Cytosolic tau aggregates are characteristic of AD and of FTD. Inhibition of autophagy (by reduction of p62 expression in mice) caused the appearance of tau aggregates in non-transgenic mice. Therefore, it was postulated that stimulation of autophagy by LNK-754-TS treatment (which upregulates p62 expression (FIG. 8e)), could reduce tau aggregates in tau transgenic mice.

Tau transgenic mice accumulate the disease-associated form of abnormally phosphorylated tau (measured by antibody AT180) in the amygdala. These mice were treated with LNK-754-TS (0.09 mg/kg, once every 24 hours) for three months. A significant reduction of phosphorylated-tau (AT180) immunoreactivity as compared to vehicle-treated mice was observed (FIG. 10). Total tau, also measured immunohistochemically (HT7), was not significantly reduced by LNK-754-TS treatment (FIG. 10).

### LNK-754-TS Normalizes Tau-Dependent Behavior in Tau Transgenic Mice.

The tau transgenic mice exhibited a pathological depressed phenotype, as measured by the forced swim task (depressed mice struggle less and float more than WT mice) (FIG. 10b). This phenotype has also been produced in normal mice that do not overexpress tau, by inhibiting autophagy (via reduction of p62 expression). LNK-754-TS treatment (0.09 mg/kg, once every 24 hours) significantly ameliorated the depressed phenotype by decreasing floating behavior and increasing struggling behavior as compared to vehicle-treated animals. Remarkably, LNK-754-TS treated mice behaved similarly to non-tg mice (FIG. 10b).

### LNK-754-TS Reduces Cognitive Deficits in a Double Transgenic Mouse Model of Alzheimer's Disease

Although extracellular amyloid plaques define the AD brain and contain a vast majority of the total Aβ in brain, a small portion of total Aβ is cytosolic and presumably aggregated and may be a primary driver of the disease process (LaFerla, F. M., et al. Nat Rev Neurosci 8, 499-509 (2007)). These cytosolic Aβ species may be autophagy substrates; stimulation of autophagy in an APP/PS1 transgenic mouse by overexpression of virally-encoded beclin caused reduction of intracellular A13. Furthermore, these intracellular Aβ aggregates may promote pathogenesis via cytosolic tau; reduction of tau expression in an APP/PS1 transgenic mouse reduced Aβ-dependent cognitive deficits, though no change in Aβ was measured (Roberson, E. D., et al. Science 316, 750-754 (2007)). The effect of LNK-754-TS treatment was investigated on a well-characterized APP/PS1 double transgenic mouse model of AD that exhibits an age- and transgene-dependent cognitive loss (Moechars, D., et al. J Biol Chem 274, 6483-6492 (1999)).

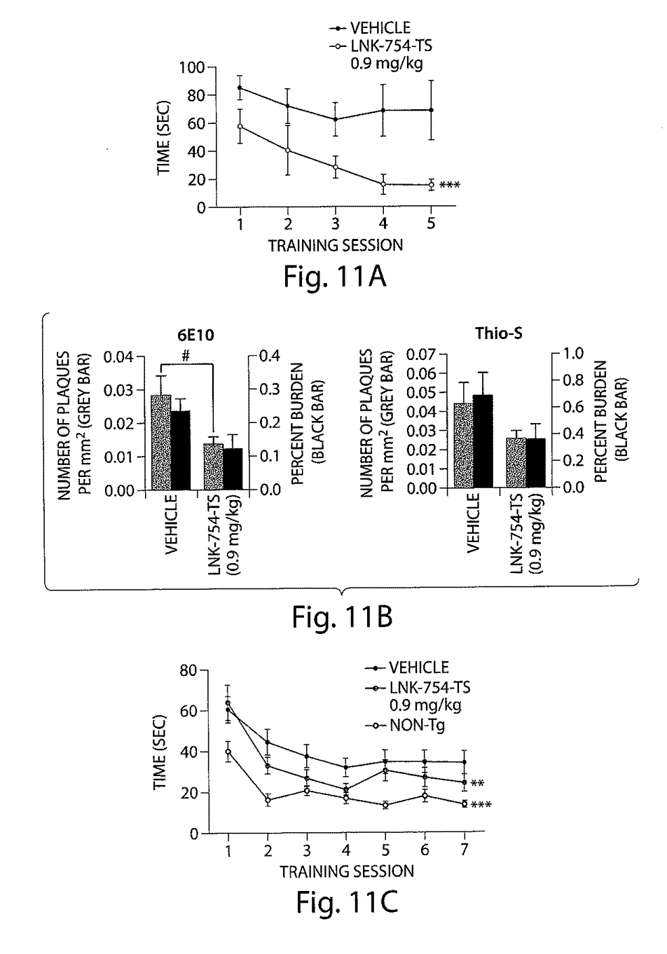

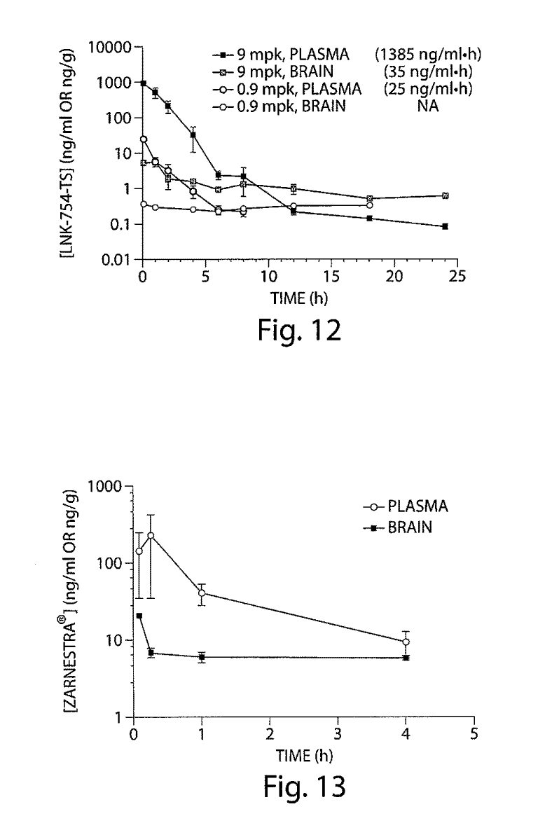

Mice were treated with LNK-754-TS for two months, tested for performance in the Morris water maze (MWM), and then sacrificed for immunohistochemical (Aβ immunoreactivity) and biochemical (ELISA measurement of Aβ40 and Aβ42) analysis. LNK-754-TS treated mice (0.9 mg/kg, once every 24 hours) performed significantly better than vehicle-treated mice in the MWM test (FIG. 11a).

In contrast to the large and significant improvement in cognition, there was a lesser, but still significant, effect on the number of Aβ (anti-amyloid 6E10) immunoreactive plaques in the area of the subiculum (FIG. 11b). There were no statistically significant changes in Thioflavin-S (Thio-S) staining in the subiculum (FIG. 11b) or in levels of Aβ40/Aβ42 extracted from whole brain fractions measured by Elisa.

In an effort to further explore the role of LNK-754-TS on the cognitive pathology in APP—PS1 mice, a cohort of the mice were treated with LNK-754-TS (0.9 mg/kg) for a much shorter period (12 days). Under these conditions, there was also a significant cognitive improvement in the LNK-754-TS treated group (FIG. 11c), but with no significant reduction in Aβ40 or Aβ42 levels, Aβ immunoreactivity or Thio-S staining. The striking results of this trial are consistent with the proposed mechanism of action (autophagy stimulation), which has the potential to clear pre-existing intracellular Aβ and tau aggregates in addition to inhibiting ongoing aggregate accumulation.

In order to rule out the possibility that the rapid observed improvement in cognition described above arose from an alternative, transgene-independent mechanism, aged non-transgenic rats (22 months old) were treated with LNK-754-TS (0.3 mg/kg and 0.9 mg/kg, once every 24 hours) and their cognitive performance was measured by MWM and compared to that of younger rats (3 months old) of the same strain. Vehicle-treated aged rats demonstrated a learning curve in both the cued and place learning phases, but were significantly impaired in terms of path length and latency to platform when compared to the vehicle-treated young group. Treatment of aged rats with LNK-754-TS yielded no significant cognitive improvement, either in the place learning curves or in either of the 2 probe tests.

Finally, it is important to note that LNK-754-TS had no effect on APP processing and secretion in a cell culture model of pathogenic Aβ production (Selkoe, D. J., at al. Ann N Y Acad Sci 777, 57-64 (1996)). In addition, LNK-754-TS treatment (0.9 mg/kg once every 24 hr for three months) in the h-APPₛ₁ transgenic mouse, which exhibits no measurable behavior pathological phenotype, did not significantly reduce the amount of cortical Aβ immunoreactivity or the amount of Aβ extracted in the insoluble fractions, which contained the vast majority of Aβ40 and Aβ42. However, a small reduction in the amounts of more soluble Aβ42 species was measured, consistent with the notion that cytosolic Aβ oligomers, rather than extracellular plaques, are autophagy substrates.

### Example 10

### Pharmacokinetics in Mice

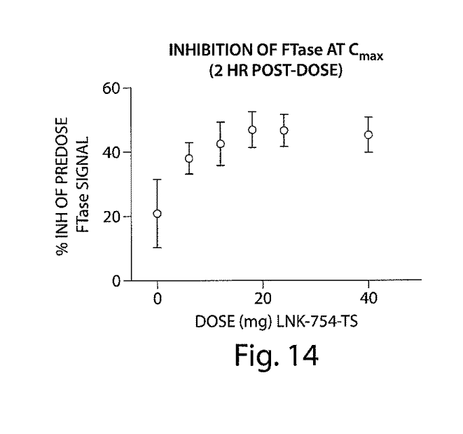

The pharmacokinetic profiles of LNK-754-TS and Zarnestra® were analyzed using methods known in the art. The results are shown in FIGS. 13-14 and the tables below. Table 3A below shows selected pharmacokinetic parameters of Zarnestra® in C57BL/6 mice plasma and brain following oral administration at dose of 5 mg/kg.

TABLE 3A

| | Pharmacokinetic Parameters | | | | | |
|---|---|---|---|---|---|---|
| | AUC₍₀₋ₜ₎ | AUC_{(0-∞)} | MRT_{(0-∞)} | t_{1/2} | Tₘₐₓ | Cₘₐₓ |
| | ng/mL * h | ng/mL * h | h | h | h | ng/mL |
| Blood | 130.23 | 131.26 | 1.76 | 1.31 | 1.00 | 40.44 |
| | ng/g * h | ng/g * h | h | h | h | ng/g |
| Brain | 44.50 | 86.86 | 11.37 | 8.10 | 1.00 | 6.84 |

Table 4A below shows selected pharmacokinetic parameters of LNK-754-TS in C57BL/6 mice following oral administration.

TABLE 4A
Selected pharmacokinetic parameters of LNK-754-TS in C57BL/6 mice
following oral administration.

| | AUC₍₀₋ₜ₎ | AUC_{(0-∞)} | MRT_{(0-∞)} | t_{1/2} | | |
|---|---|---|---|---|---|---|
| Treatment | μg/L * hr | μg/L * hr | hr | hr | Tₘₐₓ | Cmax |
| Group 5 | 729.67 | 751.99 | 2.38 | 1.50 | 1.00 | 318.41 |
| 9 mg/kg | | | | | | |
| SID) | | | | | | |
| Group 6 | 2099.01 | 2287.51 | 2.67 | 5.04 | 1.00 | 1385.64 |
| (9 mg/kg | | | | | | |
| BID) | | | | | | |
| Group 9 | 2628.78 | 2633.64 | 1.43 | 0.62 | 1.0 | 1485.63 |
| (9 mg/kg | | | | | | |
| Day 5) | | | | | | |

### Example 11

### Phase I Pharmacodynamic Analysis

Samples from a clinical study of LNK-754-TS were analyzed to measure FTase activity using SPA technology to measure the amount of ³H—FPP incorporation into a synthetic acceptor peptide after incubation in PBMC lysate. FTase substrate modification was determined using a Western blot method to determine HDJ-2 protein farnesylation state by alterations in electrophoretic migration rate. The same PBMC lysate from each patient was used from SPA and Western blot. The patient cohorts assessed were: cohort 1 (6 mg), 2 (12 mg), 2A (18 mg), 3 (24 mg), and 4 (40 mg) have been assessed. Two 8-mL blood draws supply two individual PBMC pellets after processing. These are kept separate to provide a back-up pellet in case of shipment or analytical failure. The primary samples from all cohorts were analyzed. The SPA reaction (Lysate, ³H—FPP, biotinylated acceptor peptide) is incubated at room temperature for 120 minutes and then stopped with 250 mM EDTA. Reaction progress is measured by incorporation of ³H—FPP into the peptide substrate and scintillation upon co-localization of ³H and the SPA beads via biotin-streptavidin binding. FIG. 14 shows a summary of FTase inhibition at Cmax (2 hours post dose) vs. dose of LNK-754-TS. *Mean % inhibition includes select values from the low-conc lysates.

### Example 12

### Selectivity of FTase over GGTase

Based on the use of farnesyl transferase inhibitors in treating cancer, the adverse side effects resulting from the administration of farnesyl transferase inhibitors are thought to be due to these compounds' cross reactivity with geranylgeranyl transferase (GGTase). Farnesyl transferase inhibitors that are more selective for FTase as compared to GGTase have less adverse side effects than those which inhibit both FTase and GGTase. As reported by End et al. in Cancer Research (61:131-137, Jan. 2001; Exhibit 1), tipifarnib is over 5,000 times more selective for FTase than GGTase (IC50 s of 0.86 nM and 7.9 nM for the inhibition of the farnesylation of lamin B and K-RasB peptide substrates, respectively; only 40% inhibition of the geranylgeranylation of lamin B peptide substrate by GGTase was observed at 50 micromolar). Other farnesyl transferase inhibitors such as BMS-214662 and L-778 exhibit much less selectivity for FTase. BMS-214662 exhibits a 1000-fold difference between FTase inhibitory activity and GGTase inhibitory activity (IC50 of 1.3 nM (H-Ras) or 8.4 nM (K-Ras) for FTase as compared to an 1050 of 1.9 micromolar (K-Ras) or 1.4 micromolar (H-RasCVLL) for GGTase (Cancer Res., 61:7507-16, 2001). L-778123 only exhibits a 50-fold difference between FTase inhibitory activity versus GGTase inhibitory activity (IC₅₀ of 2 nM for FTase as compared to an IC₅₀ of 100 nM for GGTase (K-Ras peptide: J. Biol. Chem. 276:24457-65, 2001).

The selectivity of LNK-754 for FTase over GGTAse is shown below in Table 5A.

TABLE 5A
Selectivity of LNK-754 for FTase over GGTase

| | H-Ras | | K-Ras | |
|---|---|---|---|---|
| | CAAX | K-Ras | CAAX | |
| H-Ras protein | Mutant | protein | Mutant | Ki FTase |
| CLVS | | CVIM | | |
| in vitro | | in vitro | | |
| FTI | GGTI | FTI | GGTI | |
| 0.9 nM | 552 nM | 72 nM | 2888 nM | |
| GGTI/FTI | 580 | GGTI/FTI | 40.11 | <0.05 nM |

What is claimed is:

1. A method of treating a proteinopathic subject, wherein the method comprises administering a compound selected from: 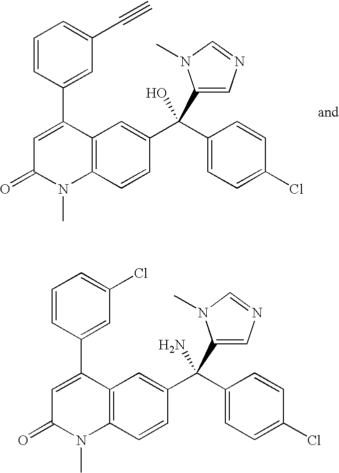
or a pharmaceutically acceptable salt thereof, to the subject in an amount that ranges from approximately 0.1 mg per day to approximately 50 mg per day.

2. The method according to claim 1, wherein the amount the compound or a pharmaceutically acceptable salt thereof, ranges from approximately 0.5 mg per day to approximately 30 mg per day.

3. The method according to claim 2, wherein the amount of the compound or a pharmaceutically acceptable salt thereof, ranges from approximately 4 mg per day to approximately 20 mg per day.

4. The method according to claim 1, wherein the amount of the compound or a pharmaceutically acceptable salt thereof, is not sufficient to inhibit the farnesylation of Ras in the brain by more than about 50%.

5. The method according to claim 1, wherein the amount of the compound or a pharmaceutically acceptable salt thereof, is sufficient to inhibit the farnesylation of UCH-L1.

6. The method according to claim 1, wherein the pharmaceutically acceptable salt administered is the D-tartrate salt of

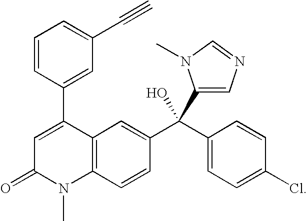

7. The method according to claim 1, wherein the proteinopathic subject is suffering from a neurodegerative disease, a cognitive impairment, a lysosomal storage disease, an ocular disease, an inflammatory disease, a cardiovascular disease, or a proliferative disease.

8. The method according to claim 7, wherein the neurodegenerative disease is selected from Parkinson's disease, diffuse Lewy body disease, multiple system atrophy, pantothenate kinase-associate neurodegeneration, amyotrophic lateral sclerosis, Huntington's disease, and Alzheimer's disease.

9. The method according to claim 1, further comprising administering to the subject a therapeutically effective amount of a non-farnesyl transferase inhibitor.

10. The method according to claim 9, wherein the non-farnesyl transferase inhibitor is selected from the group consisting of dopamine agonists, DOPA decarboxylase inhibitors, dopamine precursors, monoamine oxidase blockers, cathechol O-methyl transferase inhibitors, anticholinergics, acetylcholinesterase inhibitors, activators of neurotrophic receptors, gamma-secretase inhibitors, PDE10 inhibitors, and NMDA antagonists.

11. The method according to claim 1, wherein the subject is a human.

12. A pharmaceutical composition for treating a proteinopathic subject comprising approximately 0.1 mg to approximately 50 mg of a compound selected from 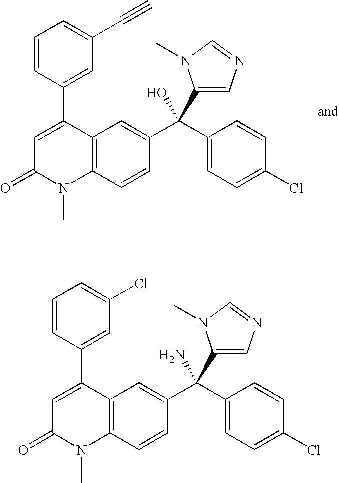
or a pharmaceutically acceptable salt thereof, and a pharmaceutically acceptable excipient.

13. The pharmaceutical composition according to claim 12 comprising approximately 0.5 to approximately 30 mg of the compound or a pharmaceutically acceptable salt thereof.

14. The pharmaceutical composition according to claim 13 comprising approximately 4 to approximately 20 mg of the compound or a pharmaceutically acceptable salt thereof.

15. The pharmaceutical composition according to claim 12, wherein the pharmaceutically acceptable salt is the D-tartrate salt of

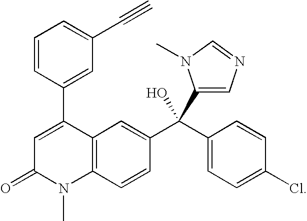

16. The pharmaceutical composition according to claim 12, wherein the proteinopathic subject is suffering from a neurodegerative disease, a cognitive impairment, a lysosomal storage disease, an ocular disease, an inflammatory disease, a cardiovascular disease, and a proliferative disease.

17. The pharmaceutical composition according to claim 16, wherein the neurodegenerative disease is selected from Parkinson's disease, diffuse Lewy body disease, multiple system atrophy, pantothenate kinase-associate neurodegeneration, amyotrophic lateral sclerosis, Huntington's disease, and Alzheimer's disease.
